(19) United States
(12) Patent Application Publication
(10) Pub. No.: US 2026/0031241 A1
(43) Pub. Date: Jan. 29, 2026

(54) GENERATION OF AN EFFICACY INDEX FOR A MEDICAL TREATMENT

(71) Applicant: IMX, Inc., Las Vegas, NV (US)

(72) Inventors: JAMES R. PLANTE, Las Vegas, NV (US); NOAH JASON SHEN, Las Vegas, NV (US); SRI GURUDATH NIKHIL NAMBURI, Las Vegas, NV (US); MOHAMAD EJAZ CHAUHAN, Las Vegas, NV (US)

(21) Appl. No.: 19/278,424

(22) Filed: Jul. 23, 2025

Related U.S. Application Data
(60) Provisional application No. 63/674,659, filed on Jul. 23, 2024.

(51) Int. Cl. G16H 50/70; G06Q 40/08; G16H 10/60

(52) U.S. Cl. CPC G16H 50/70; G06Q 40/084; G16H 10/60

(57) ABSTRACT

Techniques for generating an efficacy index that statistically reflects an efficacy of a treatment used to address a medical condition are disclosed. A service identifies a transition timestamp reflective of when a patient begins the treatment. The service accesses a first set of claims associated with the patient. The service determines a static claim metric for the patient's medical condition using a total number of claims included in the first set of claims and certain timestamp data. The service accesses a second set of claims associated with the patient's medical condition. The service determines dynamic claim metrics for the patient's medical condition using the second set of claims and other timestamp data. The service generates a patient-level index based on the static claim metric and the dynamic claim metrics. The service aggregates the patient-level index with other patient-level indexes to generate an overall efficacy index for the treatment.

### CROSS-REFERENCE TO RELATED APPLICATION

This application claims priority to U.S. Provisional Application No. 63/674,659, filed Jul. 23, 2024, which is incorporated herein by this reference.

### BACKGROUND

The price of many commodities can fluctuate significantly within a relatively short period of time. For instance, oil prices rose from $18.50 per barrel in January 1999 to over $147 per barrel in June 2008. A futures contract can help stabilize the market for a particular good.

A futures market (or simply “future”) is essentially an auction-based market that allows participants to buy and sell goods for delivery at a future point in time. That is, a future is an exchange-traded derivative contract that locks in the future delivery of an item for a price that is set today.

Prior to the availability of the futures market, oil was a one-sided market in that the oil producers set the price, and the consumers were forced to pay that prior. Once a futures market was developed for the oil industry, the price of oil generally stabilized.

Healthcare is currently a one-sided market because the payers generally have complete control over the terms. The cost of healthcare has been rising at a significant rate in recent years. Furthermore, the healthcare industry generally recognizes a vulnerability with regard to price shocks from new pharmaceuticals.

As an example, several new treatments for specific diseases have been approved, where these treatments often extend and improve the quality of a patient's life. Those treatments, however, are very expensive. As an example, consider the drug Sovaldi. Sovaldi is designed to treat and even cure hepatitis C. This drug can cost about $40,000 for a full treatment. The cost of treating hepatitis C prior to Sovaldi was about $500,000 for a full treatment.

From the insurance company's perspective, the insurance company would much rather pay $40,000 as compared to $500,000 to treat hepatitis C. Creating a futures market would provide equal footing to providers, treatment manufacturers, and ultimately patients. A futures market would allow manufacturers to sell treatments (e.g., drugs) into a futures market before those treatments were even manufactured. Doing so would greatly reduce the cost of the treatment.

A futures market typically relies on a so-called market “index.” An index is a hypothetical investment portfolio that generally represents the holdings of a portion of a financial market. Participants in the futures market rely on the index when considering whether to buy or sell a futures contract because the index helps evaluate the cost of a good.

Futures contracts are available for many goods, such as oil, because a robust index has been created for such commodities. For healthcare, however, there is no index and thus there is no futures market in the healthcare industry. A futures market in the healthcare industry could significantly help stabilize the ever increasing costs of healthcare. To achieve the futures market, however, an index is needed for the cost of healthcare.

To be complete, the index should account not only for the services that are expended when treating a disease but also for the drugs or pharmaceuticals that are prescribed and used to treat a disease. There are various techniques for linking a pharmaceutical to a medical condition.

One technique (i.e. a naïve technique) involves identifying all of the patients who have been diagnosed with a medical condition and then subsequently identifying all of the pharmaceuticals that those patients are taking. Those pharmaceuticals could then be ranked based on frequency or perhaps popularity of use. If a drug has a sufficiently high popularity, then that drug can be linked with the medical condition as either a primary drug for that medical condition or a drug used to treat a co-morbidity or associated disease that is often linked with the primary medical condition. Such an approach is wrought with false positives and false negatives. This approach also introduces a significant amount of noise into the analysis because not all drugs that a person who has a medical condition is taking may be geared specifically for that medical condition. For instance, a diabetic person might take medicine to treat diabetes, but that person might also take other medicine to treat other ailments.

Another technique involves a statistical-based analysis. This technique generally involves analyzing each drug and determining a likelihood of seeing a particular drug among patients who have a particular medical condition. The analysis also includes evaluating the likelihood of seeing that drug being used by patients who have not been diagnosed with the medical condition. If the drug is rarely (if ever) used by patients that do not have the medical condition, then there is a reasonably high likelihood that the drug is used to treat the medical condition. On the other hand, if the drug is frequently used even by patients who do not have the medical condition, then there is less certainty as to whether that drug used to treat the medical condition was effective.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

In some aspects, the techniques described herein relate to a method for generating an overall efficacy index that statistically reflects an efficacy of a particular treatment used to treat a medical condition, said method including: identifying a transition timestamp reflective of when a patient begins a particular treatment for a medical condition of the patient; accessing a first set of claims associated with the patient, the first set of claims being claims related to the patient's medical condition prior to the transition timestamp, wherein each claim in the first set of claims is associated with a corresponding timestamp, a combination of which constitutes a first set of timestamps, and wherein the first set of timestamps all pre-date the transition timestamp; determining a static claim metric for the patient's medical condition using (i) a total number of claims included in the first set of claims and (ii) a number of days spanning between an earliest timestamp included among the first set of timestamps and the transition timestamp; accessing a second set of claims associated with the patient's medical condition, the second set of claims being claims related to the particular treatment the patient performed to address the patient's medical condition, wherein each claim in the second set of claims is associated with a corresponding timestamp, a combination of which constitutes a second set of timestamps, and wherein the second set of timestamps all post-date the transition timestamp; determining a plurality of dynamic claim metrics for the patient's medical condition using (i) the second set of claims, (ii) the second set of timestamps, and (iii) the transition timestamp; generating a patient-level index based on the static claim metric and the plurality of dynamic claim metrics; and aggregating the patient-level index with a plurality of other patient-level indexes associated with the same particular treatment for the medical condition to generate an overall efficacy index for the particular treatment, the overall efficacy index statistically reflecting an efficacy of the particular treatment for the medical condition.

In some aspects, the techniques described herein relate to a computer system that generates an overall efficacy index that statistically reflects an efficacy of a particular treatment used to treat a medical condition, said computer system including: one or more processors; and one or more hardware storage devices that store instructions that are executable by the one or more processors to cause the computer system to: identify a transition timestamp reflective of when a patient begins a particular treatment for a medical condition of the patient; access a first set of claims associated with the patient, the first set of claims being claims related to the patient's medical condition prior to the transition timestamp, wherein each claim in the first set of claims is associated with a corresponding timestamp, a combination of which constitutes a first set of timestamps, and wherein the first set of timestamps all pre-date the transition timestamp; determine a static claim metric for the patient's medical condition using (i) a total number of claims included in the first set of claims and (ii) a number of days spanning between an earliest timestamp included among the first set of timestamps and the transition timestamp; access a second set of claims associated with the patient's medical condition, the second set of claims being claims related to the particular treatment the patient performed to address the patient's medical condition, wherein each claim in the second set of claims is associated with a corresponding timestamp, a combination of which constitutes a second set of timestamps, and wherein the second set of timestamps all post-date the transition timestamp; determine a plurality of dynamic claim metrics for the patient's medical condition using (i) the second set of claims, (ii) the second set of timestamps, and (iii) the transition timestamp; generate a patient-level index based on the static claim metric and the plurality of dynamic claim metrics; and aggregate the patient-level index with a plurality of other patient-level indexes associated with the same particular treatment for the medical condition to generate an overall efficacy index for the particular treatment, the overall efficacy index statistically reflecting an efficacy of the particular treatment for the medical condition.

In some aspects, the techniques described herein relate to one or more hardware storage devices that store instructions that are executable by one or more processors to cause the one or more processors to: identify a transition timestamp reflective of when a patient begins a particular treatment for a medical condition of the patient; access a first set of claims associated with the patient, the first set of claims being claims related to the patient's medical condition prior to the transition timestamp, wherein each claim in the first set of claims is associated with a corresponding timestamp, a combination of which constitutes a first set of timestamps, and wherein the first set of timestamps all pre-date the transition timestamp; determine a static claim metric for the patient's medical condition using (i) a total number of claims included in the first set of claims and (ii) a number of days spanning between an earliest timestamp included among the first set of timestamps and the transition timestamp; access a second set of claims associated with the patient's medical condition, the second set of claims being claims related to the particular treatment the patient performed to address the patient's medical condition, wherein each claim in the second set of claims is associated with a corresponding timestamp, a combination of which constitutes a second set of timestamps, and wherein the second set of timestamps all post-date the transition timestamp; determine a plurality of dynamic claim metrics for the patient's medical condition using (i) the second set of claims, (ii) the second set of timestamps, and (iii) the transition timestamp; generate a patient-level index based on the static claim metric and the plurality of dynamic claim metrics; and aggregate the patient-level index with a plurality of other patient-level indexes associated with the same particular treatment for the medical condition to generate an overall efficacy index for the particular treatment, the overall efficacy index statistically reflecting an efficacy of the particular treatment for the medical condition.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIG. 1 illustrates an example computing architecture for generating a medical treatment index.

FIG. 2 illustrates an example timeline illustrating various different treatment events.

FIG. 3 illustrates an example table showing different claims.

FIG. 4 illustrates an efficacy of a drug.

FIG. 5 illustrates an overall efficacy index.

FIG. 6 illustrates a patient-level index.

FIG. 7 illustrates behavioral patterns of an index.

FIGS. 8A and 8B illustrate further behavioral patterns.

FIGS. 9A, 9B, 9C, and 9D illustrate various different flowcharts of example methods for generating an index.

FIGS. 10 through 23 illustrate various supporting charts reflecting study results.

FIG. 24 illustrates an example computer system that can be configured to perform any of the disclosed operations.

### DETAILED DESCRIPTION

As mentioned earlier, futures and indexes are valuable tools that help to lower prices while improving product quality. Historically, an index has not been available for the medical community. The disclosed embodiments are beneficially directed to various techniques for generating an index for the medical community. While a majority of this disclosure is focused on a scenario involving an index for a drug/pharmaceutical, it should be noted how similar indexes can be generated for medical procedures, medical providers, or any medical related field. Thus, the disclosed principles should be viewed in an expansive nature and should not be viewed as being limited only to a pharmaceutical scenario.

By providing the disclosed indexes, the disclosed embodiments bring about numerous benefits, advantages, and practical applications to the medical community. In particular, practice of the disclosed principles will not only help stabilize costs within the medical community, but they will also help reduce those costs overtime. The disclosed embodiments help identify pharmaceuticals that are particularly successful in how they operate. Stated differently, it is often the case that a drug is considered “successful” if it puts itself out of work. In other words, if the drug helps cure or otherwise alleviate a patient's issues to the point where the patient no longer is reliant on a drug, then that drug is likely to be considered a successful drug. The disclosed embodiments are beneficially designed to identify the success rates of drugs. Identification of these success rates can help spur additional investment and further improvement in the drug or in related drugs to increase their success even more.

Additional benefits include advanced abilities to facilitate comparisons between different drugs, treatments, or any other technical sector in which an index is generated. Using the medical field as one example, the disclosed principles allow healthcare providers, researchers, and policymakers to compare the therapeutic benefits of different drugs objectively. This comparison is crucial in determining the most effective treatment options available for patients. The embodiments also enhance decision making. For instance, the embodiments aid in clinical decision-making by offering evidence-based data on the effectiveness of treatments. This helps clinicians choose the most appropriate medication for their patients based on the drug's efficacy.

The embodiments also enhance regulatory approval. Regulatory agencies might use the drug efficacy index as part of their criteria for approving new drugs for market entry. Thus, the embodiments can help ensure that new medications provide a significant therapeutic benefit over existing treatments.

The embodiments also improve pharmacoeconomic analysis. For instance, the disclosed principles are used in pharmacoeconomic analyses to assess the value of drugs in terms of their cost-effectiveness. A drug with a higher efficacy index might justify a higher price point if it significantly improves patient outcomes.

The embodiments also enhance research and development. For pharmaceutical companies, the efficacy index can guide the development of new drugs. Advantageously, the embodiments can help identify areas where there is a need for more effective treatments and can drive innovation towards meeting these needs. Policy and formulary decisions are also improved. For instance, healthcare policymakers and insurance companies can use the efficacy index to make informed decisions about drug coverage and reimbursement, ensuring that only the most effective and beneficial drugs are supported. Accordingly, these and numerous other benefits will now be described in more detail throughout the remaining portions of this disclosure.

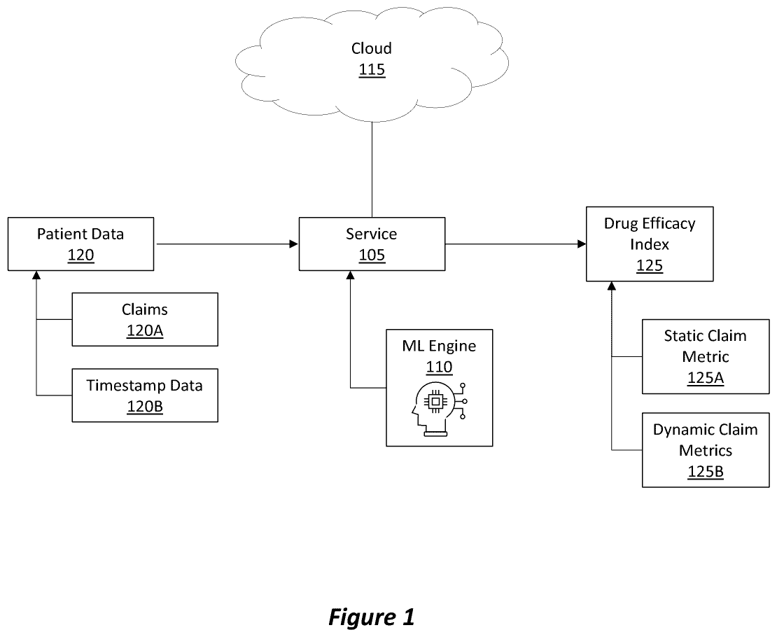

Attention will now be directed to FIG. 1, which illustrates an example computing architecture 100 in which the disclosed principles can be employed. Architecture 100 includes a service 105, which can be implemented by any type of computing device. As used herein, the term “service” refers to an automated program that is tasked with performing different actions based on input. In some cases, service 105 can be a deterministic service that operates fully given a set of inputs and without a randomization factor. In other cases, service 105 can be or can include a machine learning (ML) or artificial intelligence engine, such as ML engine 110. The ML engine 110 enables the service 105 to operate even when faced with a randomization factor.

As used herein, reference to any type of machine learning or artificial intelligence may include any type of machine learning algorithm or device, convolutional neural network(s), multilayer neural network(s), recursive neural network(s), deep neural network(s), decision tree model(s) (e.g., decision trees, random forests, and gradient boosted trees) linear regression model(s), logistic regression model(s), support vector machine(s) (“SVM”), artificial intelligence device(s), or any other type of intelligent computing system. Any amount of training data may be used (and perhaps later refined) to train the machine learning algorithm to dynamically perform the disclosed operations.

In some implementations, service 105 is a cloud service operating in a cloud 115 environment. In some implementations, service 105 is a local service operating on a local device. In some implementations, service 105 is a hybrid service that includes a cloud component operating in the cloud 115 and a local component operating on a local device. These two components can communicate with one another.

Generally, service 105 is tasked with accessing patient data 120 to generate a drug efficacy index 125. The drug efficacy index 125 is generated based on a static claim metric 125A and one or more dynamic claim metrics 125B. These metrics will be discussed in more detail later.

Typically, patient data 120 includes data from a large set of patients, such as over 100 patients. For instance, the number of patients can be 100, 200, 300, 400, 500, 1,000, 2,000, 3,000, 4,000, 5,000 or more than 5,000 or any number therebetween. The larger the dataset the more accurate the results of the index 125 will be. The patient data 120 includes claims 120A and timestamp data 120B. The claims 120A and timestamp data 120B will be discussed in more detail later.

Typically, the patient data 120 is anonymized so that no patient's personally identifiable information (PII) can be determined. Thus, service 105 operates on anonymized data that is structured in a manner so that no entity can determine any actual patient's PII.

The drug efficacy index 125 is a numerical indicator that tracks and monitors (e.g., in real-time) the efficacy or success rate of a drug. This index 125 can then be used to promote highly successful drugs or reduce the use of drugs that are not successful or do not have a threshold level of success. This index 125 is generated using various operations, and some of the remaining figures help illustrate those operations.

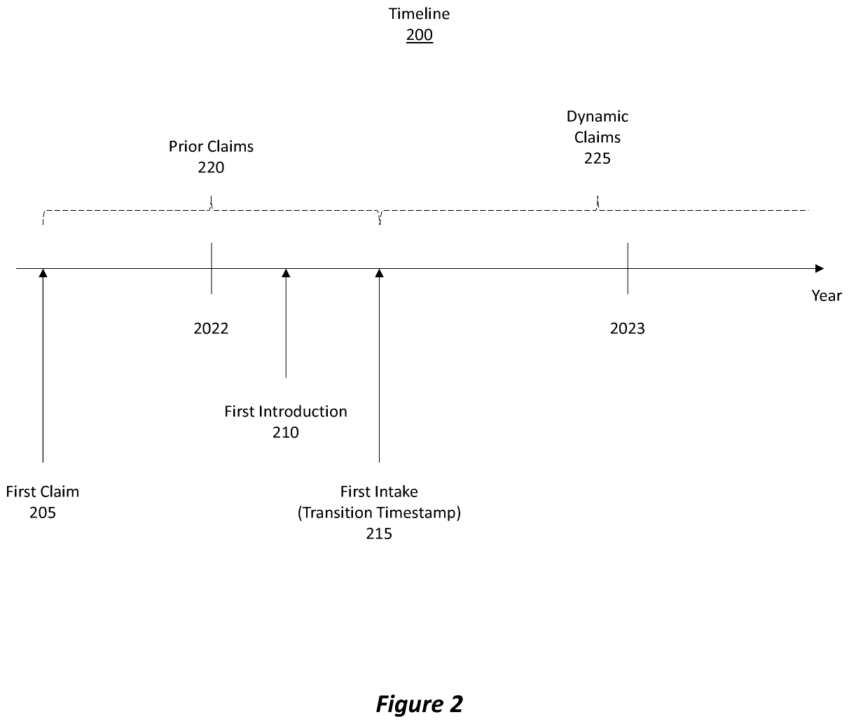

For instance, FIG. 2 shows an example timeline 200 depicting a few instances in time that are used to help generate the index 125 by service 105. In particular, timeline 200 shows how an index for the year 2023 is calculated based on certain events that started in the prior years (e.g., 2021 and 2022). It should be noted that this time delay (e.g., the index is calculated for the year 2023 even though the drug was released in 2022) is because the widescale adoption of a drug is not immediate; rather, it takes some amount of time to be “adopted” (i.e. a threshold number of individuals are using the drug to allow for adequate generation of the index). As a consequence, an index for the drug is not immediately generated. FIG. 2 generally portrays three cases: neutral, positive efficacy and inefficacy (negative difference).

Rates of adoption for a drug can vary widely. In some scenarios, the adoption time might take only about 2 months. In other scenarios, the adoption time might take 6-9 months. In some cases, it might initially appear as though a drug has been adopted within 2 months' time, but subsequently additional data is received, and the additional data reveals that the adoption time is longer (e.g., perhaps 9-12 months). Thus, different adoption rates occur for different drugs. In most scenarios, the preferred amount of time to wait until adoption has stabilized is around 12 months.

Service 105 of FIG. 1 is able to analyze patient data to identify when a patient first filed a claim for a particular ailment, as shown in FIG. 2 by first claim 205. In this example scenario, the patient filed the first claim 205 in the year 2021. In some scenarios, first claim 205 represents a scenario where the patient was first prescribed a new drug to help treat the patient's condition. In some scenarios, first claim 205 simply represents a claim or a note in the patient's medical history for the medical condition but the patient has not yet started taking a prescription for that medical condition/ailment. Thus, first claim 205 should be viewed as a first recognition of the patient having a given ailment. First claim 205 can include, but is not required to include a scenario in which the patient starts taking a drug for that ailment. This first claim 205 can be included in the patient data 120 of FIG. 1.

Subsequently, a new drug came to market in the year 2022, as shown by first introduction 210. After having already consumed (potentially) the previous drug since the time represented by first claim 205, the patient then consumes the new drug for the first time in 2022, as shown by first intake 215 (e.g., a “transition timestamp”). Additionally, or alternatively, the patient may not have been taking a drug during the time period between first claim 205 and first intake 215; instead, the patient's medical history may have simply reflected the patient's ailment during that time period. Thus, in some scenarios, the patient may have been taking a drug between first claim 205 and first intake 215 (and the patient transitioned from the first drug to the new drug) while, in other scenarios, the patient may not have been taking a drug between first claim 205 and first intake 215. Information suitable to determine that the patient either took or was prescribed the new drug at first intake 215 can also be included in the patient data 120 of FIG. 1. In most scenarios, an inference is made that the date of the new drug's prescription to the client is the date that the patient starts taking the new drug/medication.

The period of time labeled by prior claims 220 reflects the amount of time and the number of dosages (or “claims”) the client consumed of the other drug prior to consuming, for the first time, the new drug. Thus, prior claims 220 represents a baseline of the patient's health history (and in particular drug consumption) for a particular ailment prior to consuming a new drug for the first time. Stated differently, the time period between first claim 205 and first intake 215 constitutes a pre-ingestion time period with respect to the new drug, and the data for this time period includes both claim data and timestamp data. A “new” drug typically refers to a drug that is first being introduced to the market. An example will be helpful.

Suppose a patient contracts “Ailment A” in the year 2021. In response, the patient starts to take “Drug A” for Ailment A. The patient might take Drug A 4 times per month. Service 105 is able to acquire this data and determine the patient's prior drug usage for Ailment A. This prior information constitutes the patient's baseline drug response to Ailment A.

Now, a new drug (e.g., “Drug B”) for Ailment A is released in early 2022. In mid-2022, the patient tries Drug B for the first time, as represented by first intake 215. The patient continues to take Drug B, and eventually Drug B solves Ailment A, and the patient is no longer dependent on Drug B. Thus, in this scenario, Drug B was highly effective in treating Ailment A. The subsequent monitoring of Drug B is represented in FIG. 2 with the label dynamic claims 225. As will be described in more detail shortly, service 105 is able to use the prior claims 220 history as well as the dynamic claims 225 data to determine the efficacy for Drug B.

During the prior claims 220 time period, service 105 is tasked with calculating the average claims per day during that time period, where the average is for the previous drug or claims that were used during that time period. In this scenario, the average is computed by dividing the total number of claims (e.g., prescription servings) by the total number of days in that time period. Because service 105 is reliant on the average data, the duration of the pre-ingestion period can be viewed as being normalized. Therefore, regardless of whether the pre-ingestion period is two months or two years, this pre-ingestion time period can be considered as a normalized time period based on the calculations performed by service 105. This information will later be used to determine the efficacy for a given drug.

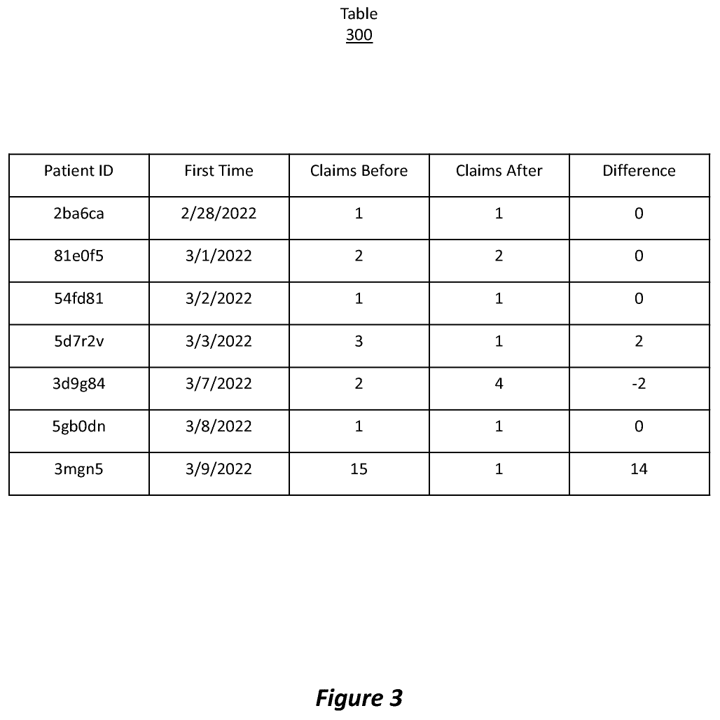

FIG. 3 shows an example table 300 that includes some data reflective of the number of claims prior to consuming a new drug versus the number of claims after starting the new drug. Table 300 can be used to help determine a drug's efficacy.

In particular, table 300 lists a number of different patients (identified using anonymized data) as well as the date these patients first started taking the new drug for the same ailment. For instance, all of the patients identified in table 300 suffer from the same ailment and have been taking the same medication for that ailment. At the date listed in table 300, each of these patients starts to take the new drug/medication.

The column labeled “Claims Before” represents the number of claims that patient had prior to that patient's corresponding “First Time” using the new drug. For instance, the first patient (labeled as patient “2ba6ca”) has one claim prior to the Feb. 28, 2022 date. On Feb. 28, 2022, the patient switched to using the new drug. Table 300 further illustrates the number of claims the patient had after the Feb. 28, 2022 date. This “Claims After” column reflects the number of claims the patient has had while using the new medication. The “Difference” column is the difference between the “Claims Before” column and the “Claims After” column. Going through each row, the values in the “Claims Before” rows are as follows: 1, 2, 1, 3, 2, 1, and 15. Similarly, the values in the “Claims After” rows are as follows: 1, 2, 1, 1, 4, 1, and 1. The differences values are as follows: 0, 0, 0, 2, −2, 0, and 14. Positive values generally reflect a scenario in which the patient seemingly was able to reduce dependence on medication to treat his/her ailment. The inference is that if the “Difference” column is a positive value, then total healthcare costs have gone down because fewer claims are being filed. Thus, the “Difference” column can provide an initial gauge on a drug's efficacy.

It should be noted how, with time, the numbers in the “Claims After” column will likely vary. For instance, it might be the case that the patient was on medication only for a select number of months prior to the “First Time.” Subsequently, however, the patient might be on the new medication for multiple years, resulting in the values in the “Claims After” increasing throughout time. In response, the “Difference” values will similarly change. Thus, the efficacy (as represented by the values in the “Difference” column) may change over time.

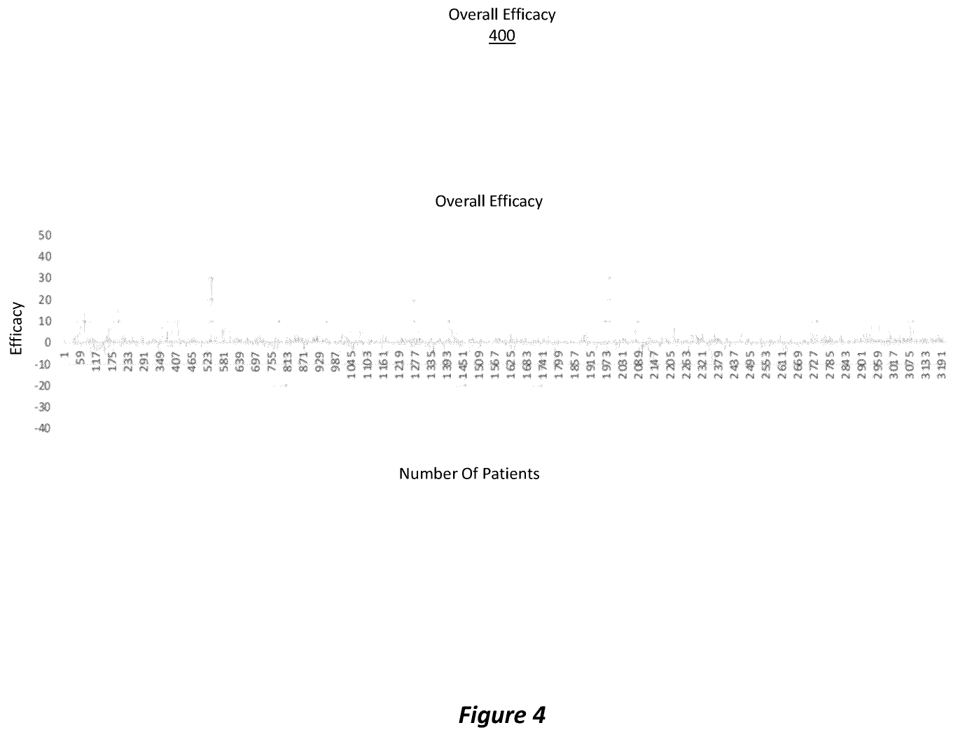

FIG. 4 shows a table outlining the overall efficacy 400 of a drug. This table is generated by averaging all of the individual efficacies (e.g., from table 300 of FIG. 3) from all of the individual patients to generate an aggregated efficacy. In FIG. 4, the x-axis represents the number of total patients, and the y-axis represents the efficacies for those patients. For instance, in this example scenario, approximately 3,191 patients are taking the same drug. These efficacies in FIG. 4 can be averaged together to generate an overall efficacy for this one drug. The overall efficacy represents the average number of claims that are filed by patients after starting consumption of the new drug. Thus, the equation for calculating average efficacy is represented by the following equation:

$$DE = \frac{1}{n} {\sum }_{0}^{n} DEPP$$

Where DE is the average drug efficacy and where DEPP is the drug efficacy per prescribed patient. The above equation is how the efficacy is calculated for a given specific day or time period. It is desirable to generate an index that represents the efficacy over a longer time period than just one day. Stated differently, the disclosed embodiments intelligently generate an index that is representative of a drug's efficacy and that represents a time period longer than just a single day.

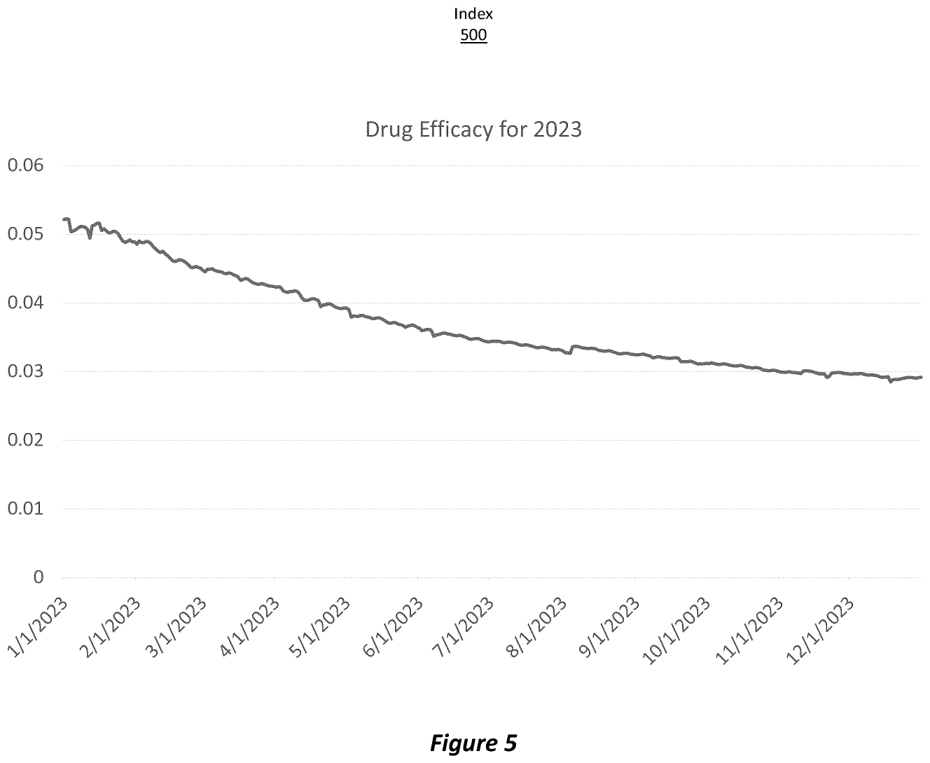

The disclosed index is generated in a manner to illustrate how, as each new day passes, the length of the post ingestion time period (i.e. the time period that starts at first intake 215 in FIG. 2) increases, and this increase has an impact on the overall efficacy of the drug. The overall efficacy is computed in a dynamic manner using the following equation:

$$DEOP = Patient ’ s Fixed Average Daily Claims - Patient ’ s Dynamic Average Daily Claims$$

Where DEOP is the drug efficacy on a patient, where the patient's fixed average daily claims is the data prior to consuming the new drug, and where the patient's dynamic average daily claims is the data after consuming the new drug. For instance, with reference to FIG. 2, the patient's fixed average daily claims corresponds to the data from the prior claims 220, and the patient's dynamic average daily claims corresponds to the data from the dynamic claims 225. The dynamic average changes every day. The embodiments compute the efficacy for a given day and for a given patient. Then, the embodiments average all of the efficacies together for that given day. This average is then included in the index that is generated for the given drug. FIG. 5 shows an example index 500 that reflects the aggregated efficacy for a given drug over a period of time.

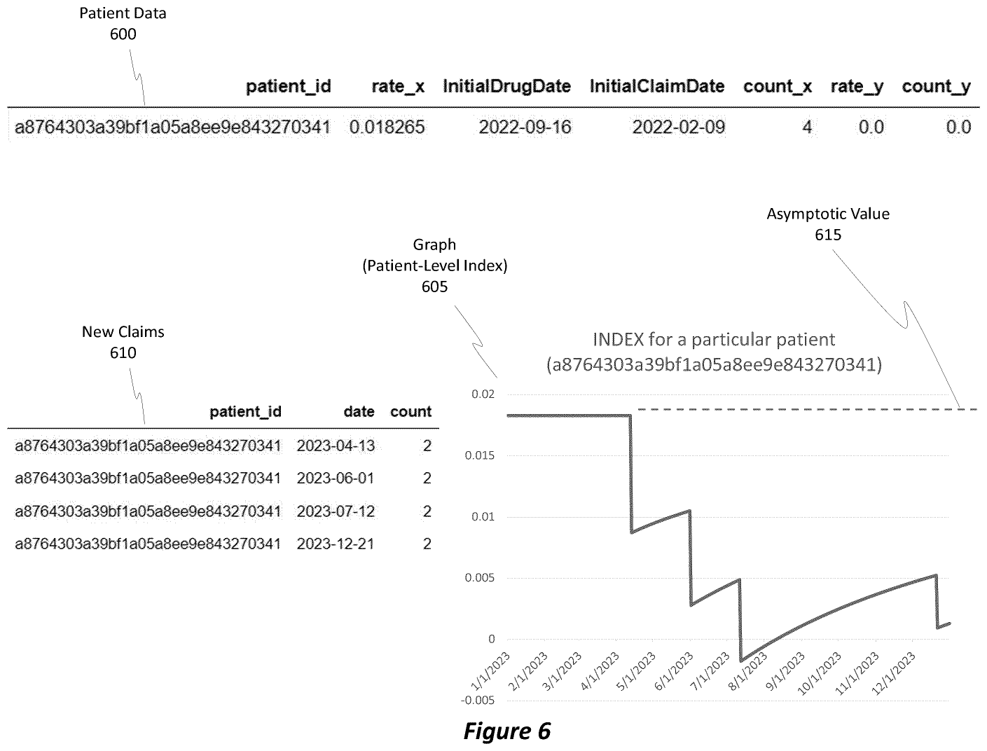

To help further understand the disclosed concepts, an example focused on a single patient will now be provided. FIG. 6 shows an example scenario involving patient data 600, which lists an anonymized identifier for the patient, the initial drug date for the patient (i.e. the date the patient first started taking the new drug, e.g., 2022-02-16), and the date of the first claim filed for the patient for a given ailment (e.g., 2022-02-09). Between the time period of 2022-02-09 and 2022-09-16 (i.e. 219 total days), the patient submitted 4 total claims for medicine, as shown by the value “4” in the “count_x” column. The average claims per day during this time period is thus 4/219 =0.018265, as shown by that value in the “rate_x” column.

In this example, and as shown by the graph 605 (i.e. a “patient-level index”), the index for this individual patient starts Jan. 1, 2023, which is approximately 3.5 months after the Sep. 16, 2022 first consumption date. During that 3.5 month time period, zero claims were submitted, as shown by the “0” value in the “count_y” column of the patient data 600. This 0 value suggests that the drug was 100% effective during that time period because the patient stopped submitting new claims. The 0 count results in a rate value of 0 as well, as shown by the “rate_y” column.

Recall, the individual patient's index is computed as the patient's fixed average daily claims (i.e. the 0.018265 value) minus the patient's dynamic average daily claims, which in this case is initially 0, as mentioned above and as shown by the 0 value in the “rate_y” column. Because the “rate_y” value is 0, the same efficacy (i.e. 0.018265) is used during the start of the index, as shown by that value reflected in graph 605 beginning on Jan. 1, 2023. This constant value indicates no variability in the number of claims that have been submitted, resulting in the index being flat and constant, and the patient did not submit any new claims after consuming the drug for the first time for at least that given time period.

It was also the case that 0 claims were submitted until 2023-04-13. As a result, the index remained constant at the 0.018265 value. Thereafter, new claims were submitted, as shown by new claims 610. To illustrate, on 2023-04-13, two claims were submitted. Similarly, two claims were each submitted on the following dates: 2023-06-01, 2023-07-12, and 2023-12-21. Filing the two claims on 2023-04-13 dynamically impacted the index, as shown by the reduction in efficacy in the graph 605 at the 2023-04-13 timestamp. The new dynamic rate is calculated as two claims over the course of approximately seven months (e.g., from Sep. 16, 2022 to Apr. 13, 2023 which is a total of 209 days). This rate then has the value of 0.009569 average claims per day. The index is updated by taking the difference between 0.018265 and 0.009569, which is the following value: 0.008696, and which is reflected in the graph at the 2023-04-13 timestamp.

Notice, after the 2023-04-13 timestamp, the graph has a curved elliptical shape. This curved shape occurs because of the dynamic manner in which the index is calculated. As each new day passes without a new claim being filed, the graph changes in an elliptical manner and also an asymptotic manner. That is, the index continues to increase in value with each new passing day while a claim has not been filed. This increase occurs because the divisor in the above equation increases daily while the dividend remains the same. This trend continues until the 2023-06-01 timestamp when two new claims are filed. Then, another drop in efficacy occurs.

At the 2023-06-01 timestamp, 258 days have now passed since 2022-09-16. During this 258 days, a total of 4 claims have now been filed. As such, the average daily claim count is 4/258=0.0155038. That value is subtracted from the 0.018265 value, resulting in the following value: 0.0027612. This value is shown in graph 605 at the 2023-06-01 timestamp.

After the 2023-06-01 timestamp, the index again starts to climb in the dynamic manner mentioned above. This climb continues until the 2023-07-12 timestamp when two additional claims are filed. Then, another drop occurs. After that drop, the index again starts to climb in the asymptotic and elliptical manner. This climb occurs until the 2023-12-21 timestamp when two more claims are filed and another drop in the index occurs. Accordingly, in this manner, an individual index (i.e. a patient-level index) can be generated for an individual patient.

Subsequently, service 105 is able to aggregate the individual indexes from any number of patients to generate an aggregated index, which was shown in FIG. 5. In this manner, the passage of time without any new claims being filed has an overall positive impact on the drug's efficacy function, and the index automatically recovers throughout time when no new claims are filed.

It should also be noted how the absolute max value of the index and also the asymptotic value 615 of the graph 605 is the initial value of the index, which, in this case, is 0.018265. As time passes without new claims being filed, the index will recover and asymptotically reach the asymptotic value 615. If new claims are filed, the index will drop in value and then subsequently recover until such time as another new claim is filed.

Service 105 is further configured to derive or infer information from the index. For instance, service 105 can review the data in the index and resolve various questions about the trend of the index. One question that service 105 can resolve is the amount of time required for the efficacy index to return to its peak value, or rather, to return to a threshold level relative to its peak value. For instance, if no subsequent claims are filed, service 105 can predict or derive the amount of time that will elapse in order for the index to substantially return to its original starting value. As mentioned previously, the index returns to the original value in an asymptotic manner, so the index will never fully recover. Service 105 can establish one or more thresholds and use those thresholds to determine the amount of time. For instance, service 105 may determine the amount of time needed for the index to return to 95% of its original starting value (or any other threshold percentage other than a true 100%). This derivation can be used to assess the drug's performance and, by extension, the drug's quality. Investors can use this index to determine when or if to invest in a given drug.

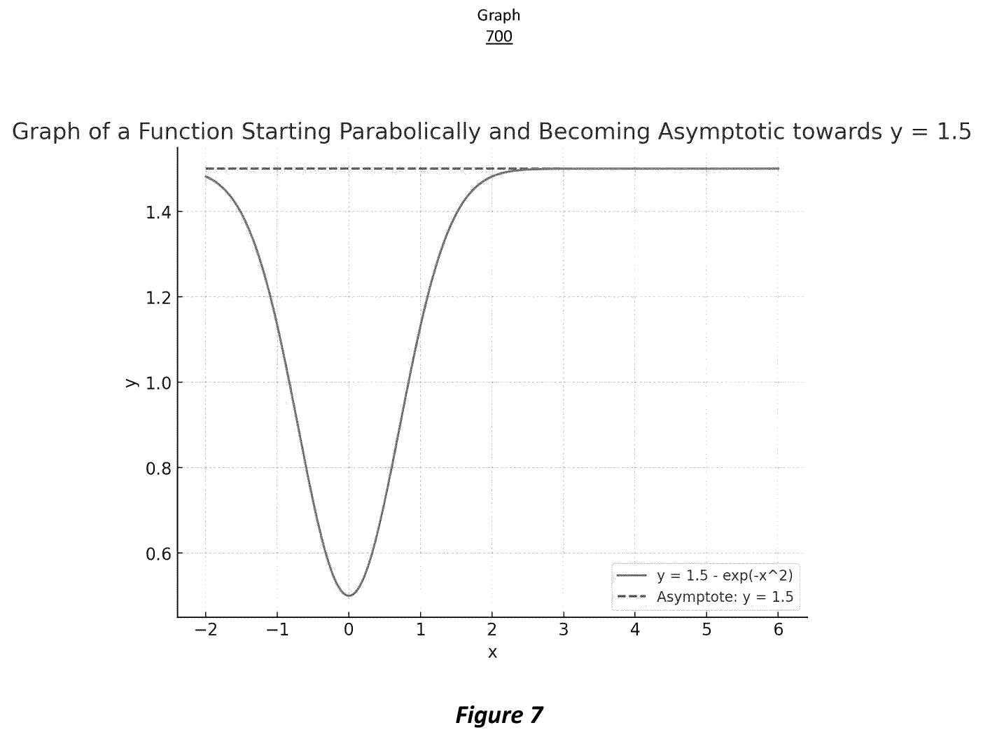

FIG. 7 shows an example graph 700 illustrating how curves can asymptotically recover. Graph 700 generally represents a behavior that the index shown in FIG. 6 will generally follow. Based on this discerned behavior, service 105 can then make various predictions and inferences as to the behavior of a drug's index. It should be noted how the depiction shown in FIG. 7 is provided simply for example purposes only and should not be viewed as limiting to the disclosed embodiments. That is, the geometry of the curve is provided simply as one example implementation.

Returning to FIG. 6, it should also be noted how the impact of submitting the same number of claims (e.g., 2 claims) has a diminishing impact over time. For instance, the first time 2 claims are submitted has the largest delta drop to the index. With each successive filing of the same number of claims, the delta drop is reduced such that the impact of subsequently filing the same number of claims has a diminishing impact on the index. The overall index 500 of FIG. 5 thus represents the entire index for the drug, and the index 500 is computed by averaging all of the individual indexes computed for all of the individual patients.

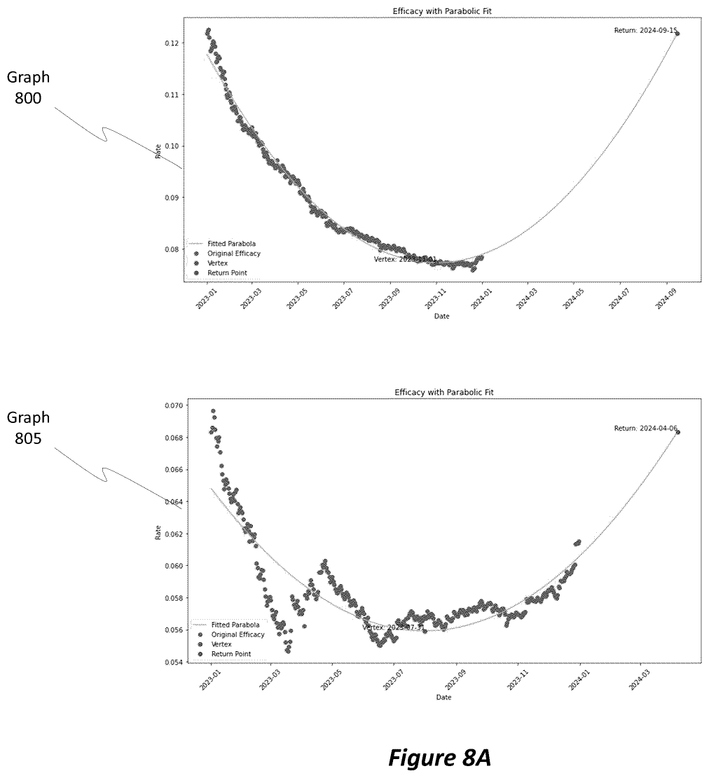

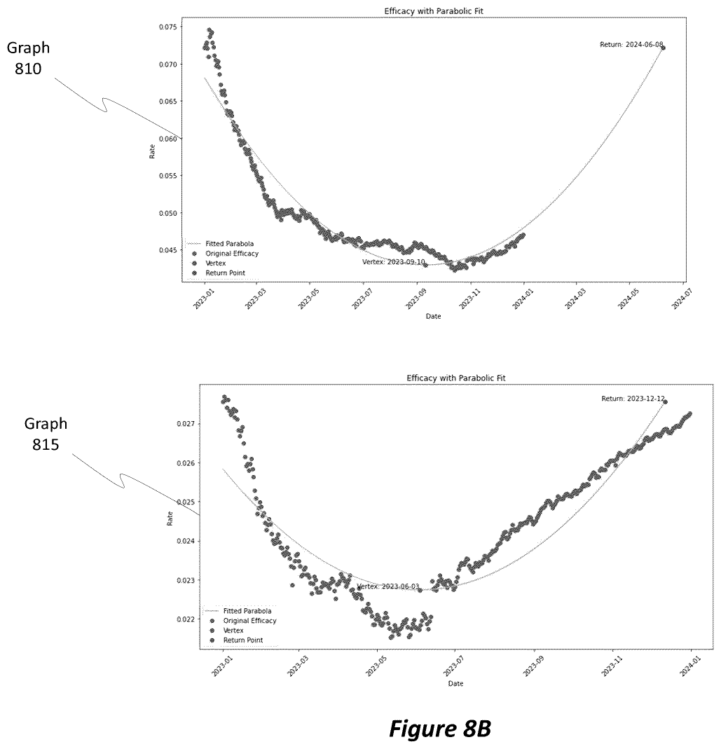

FIGS. 8A and 8B illustrate various different graphs (e.g., graph 800, graph 805, graph 810, and graph 815) that show how certain predictions can be made based on an anticipation that the behavior of the index will generally follow a parabolic or predetermined shape. As a result of having the ability to make these predictions, the index can be used as a financial predictor or an investment advisor. For instance, by assuming that the efficacy index follows a parabolic shape, service 105 can extrapolate the index's entire form based on a specified range of dates (e.g., perhaps a year) and estimate the vertex of the shape. The vertex is the lowest efficacy value of the index. By fitting the parabolic shape to the plotted index, service 105 can then extrapolate the index and estimate how long it will take for the index to fully recover, or rather, to recover to a threshold level relative to the original index value. Similarly, service 105 can predict when and what the lowest value for the index will be.

It should be noted that indexes can be computed for any drug or for any number of different drugs. These indexes can then be compared against one another to determine which drugs are most efficient. As another example, suppose a number of drugs are used to treat the same ailment. Indexes for these different drugs can be computed, and then these different indexes can be combined together (e.g., averaged) to generate an index for general treatment of the underlying ailment. As an example, suppose four different drugs are used to treat Ailment A. Service 105 can generate four different indexes, one for each of the drugs. The four different indexes can then be combined together to reflect the overall treatment efficacy for treating Ailment A based on the entire combination of drug efficacies.

The disclosed techniques for determining efficacy can be applied to any technology sector. As one example, service 105 can be configured to determine an efficacy index for a medical procedure performed to cure a given ailment. Similarly, there might be multiple different procedures that can be performed for that ailment. The individual indexes for these multiple different procedures can be averaged together to generate an overall index for treating the ailment. From this example, one will appreciate how the disclosed principles can be employed in a broad manner, and these principles are not simply limited to drugs or procedures. Thus, although FIG. 1 illustrates a “drug efficacy index,” service 105 is able to generate an index for any scenario, without limit.

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

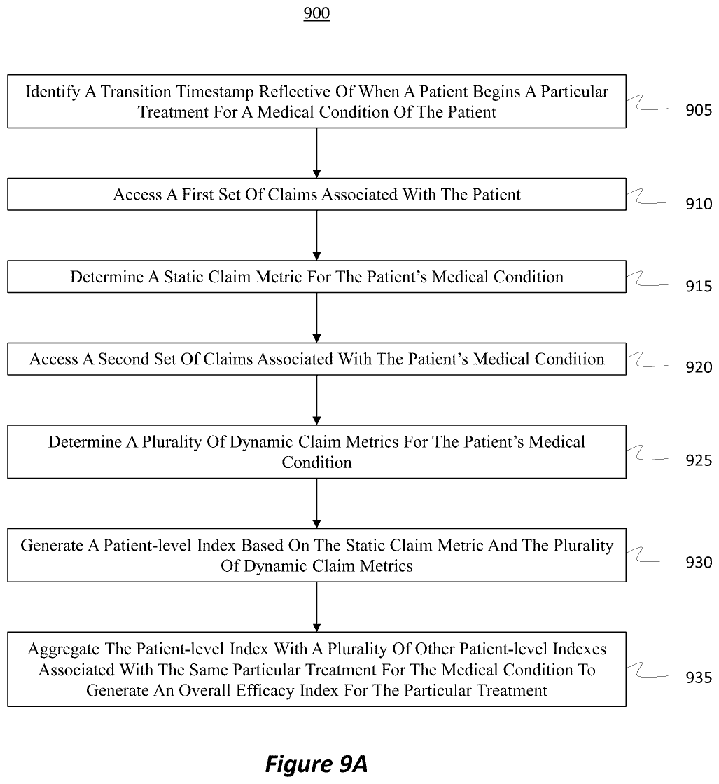

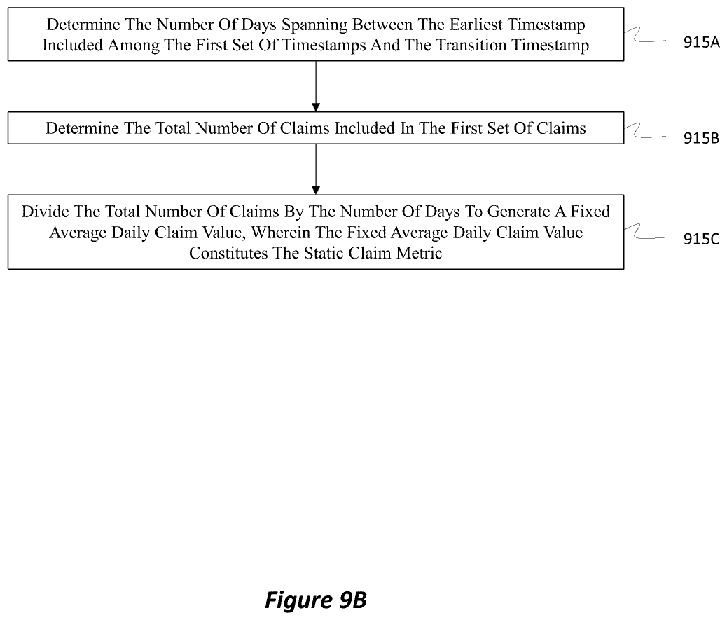

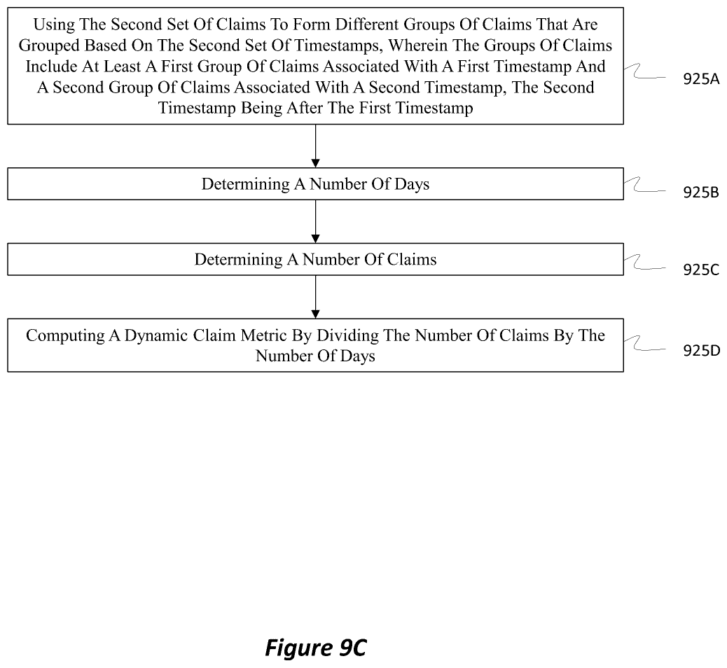

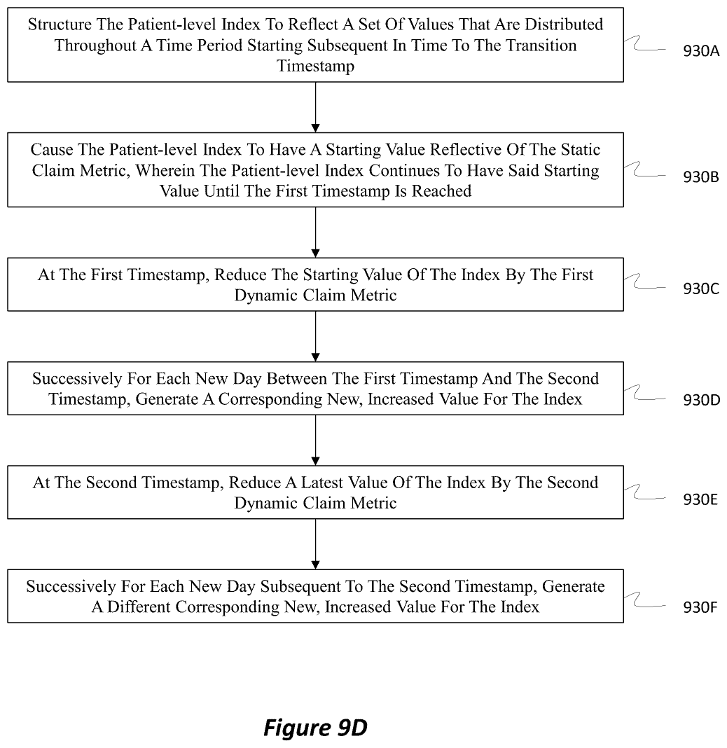

Attention will now be directed to FIG. 9A, which illustrates a flowchart of an example method 900 for generating an index for a drug/pharmaceutical. Method 900 can be implemented within architecture 100 of FIG. 1; further, method 900 can be performed by service 105. Thus, service 105 can perform method 900, which involves generating an overall efficacy index that statistically reflects an efficacy of a particular treatment used to treat a medical condition.

In some scenarios, the treatment involves a pharmaceutical such that the index reflects an efficacy of the pharmaceutical. In some scenarios, the treatment involves a medical procedure such that the index reflects an efficacy of the medical procedure.

Method 900 includes an act (act 905) of identifying a transition timestamp reflective of when a patient begins a particular treatment for a medical condition of the patient. For instance, the “transition timestamp” corresponds to the first intake 215 timestamp shown in FIG. 2. In some scenarios, identifying the transition timestamp can be performed via the use of a machine learning algorithm.

Act 910 includes accessing a first set of claims associated with the patient. For instance, the prior claims 220 in FIG. 2 corresponds to this first set of claims, and the claims 120A of FIG. 1 may include these claims. The first set of claims are claims related to the patient's medical condition prior to the transition timestamp. These claims may describe prior medications or treatments used by the patient in treating the medical condition.

Each claim in the first set of claims is associated with a corresponding timestamp. The combination of these timestamps constitutes a first set of timestamps. The first set of timestamps all pre-date the transition timestamp. These timestamps can be included in the timestamp data 120B of FIG. 1.

Act 915 includes determining a static claim metric (e.g., static claim metric 125A from FIG. 1) for the patient's medical condition using (i) a total number of claims included in the first set of claims and (ii) a number of days spanning between an earliest timestamp included among the first set of timestamps and the transition timestamp. For instance, the total number of claims are the total number of claims included in the prior claims 220 of FIG. 2. The earliest timestamp corresponds to the timestamp of the first claim 205, and the transition timestamp corresponds to the timestamp of the first intake 215. Thus, the number of days corresponds to the number of days between first claim 205 and first intake 215. FIG. 9B further clarifies act 915.

The process of determining the static claim metric is performed by the acts recited in FIG. 9B. For instance, act 915A includes determining the number of days spanning between the earliest timestamp included among the first set of timestamps and the transition timestamp. In FIG. 6, the total number of days is the number of days between 2022-02-09 and 2022-09-16.

Act 915B includes determining the total number of claims included in the first set of claims. In the example of FIG. 6, the total number of claims is 4.

Act 915C includes dividing the total number of claims by the number of days to generate a fixed average daily claim value. The fixed average daily claim value constitutes the static claim metric. The static claim metric corresponds to the starting value of the graph 605 in FIG. 6. The starting value, in this example, has a value of 0.018265.

Returning to FIG. 9A, act 920 includes accessing a second set of claims associated with the patient's medical condition. The second set of claims are claims related to the particular treatment the patient performed to address the patient's medical condition. Each claim in the second set of claims is associated with a corresponding timestamp, a combination of which constitutes a second set of timestamps. The second set of timestamps all post-date the transition timestamp. This set of claims corresponds to the claims found in the dynamic claims 225 of FIG. 2, and they can also be included among the claims 120A of FIG. 1. The new claims 610 also represents this second set of claims. Notice, the new claims 610 include claim data as well as timestamp data.

Notably, in some scenarios, the second set of timestamps/claims is not fixed like the first set of timestamps; instead, the second set of timestamps can extend to the present day. This dynamic nature of the second set of claims/timestamps provides an opportunity to assess efficacy over time. As time progresses, efficacy has the chance to emerge and consolidate further. If this second set were fixed, one could argue that the efficacy assessment is biased toward a limited portion of the treatment, leaving no room for the drug to act fully. Additionally, this dynamic aspect allows for an index that can be updated daily, with each passing day impacting the overall efficacy assessment.

Act 925 includes determining a plurality of dynamic claim metrics (e.g., dynamic claim metrics 125B of FIG. 1) for the patient's medical condition using (i) the second set of claims, (ii) the second set of timestamps, and (iii) the transition timestamp. FIG. 9C further clarifies act 925. For instance, the second set of timestamps can be those illustrated among the new claims 610 in FIG. 6.

The process of determining the plurality of dynamic claim metrics is performed by the acts recited in FIG. 9C. For instance, act 925A includes using the second set of claims to form different groups of claims that are grouped based on the second set of timestamps. In some cases, a group may include only a single claim while in other cases a group may include multiple claims. For instance, in FIG. 6, the new claims 610 are shown as being organized into groups based on timestamp data. Notice, on the date 2023-04-13, two claims are grouped together.

The groups of claims include at least a first group of claims associated with a first timestamp and a second group of claims associated with a second timestamp. The second timestamp is after the first timestamp. Acts 925B, 925C, and 925D are then repeated for the two groups.

For instance, for the first group of claims, act 925B includes determining a second number of days spanning between the transition timestamp and the first timestamp. For instance, using FIG. 6 and new claims 610 as a reference, service 105 can determine the number of days between the 2023-04-13 timestamp and the initial drug date of 2022-09-16 shown in the patient data 600.

For the first group of claims, act 925C includes determining a second number of claims included in the first group. For instance, using FIG. 6 as a reference, the number of claims for the 2023-04-13 timestamped group may be two.

For the first group of claims, act 925D includes computing a first dynamic claim metric by dividing the second number of claims by the second number of days.

As mentioned earlier, the same processes are performed for the second group of claims. It should also be noted how the index asymptotically increases between the timestamps of the first group and the second group.

For the second group of claims, act 925B includes determining a third number of days spanning between the transition timestamp (e.g., 2022-09-16 in FIG. 6) and the second timestamp (e.g., 2023-6-1 in FIG. 6).

For the second group of claims, act 925C includes determining a third number of claims included in the second group (e.g., the claim count of 2 in FIG. 6).

For the second group of claims, act 925D includes computing a second dynamic claim metric by dividing the third number of claims by the third number of days. This second dynamic claim metric is not a fixed metric and is updatable at a periodic rate. For instance, this metric gives rise to a daily updatable index.

Returning to FIG. 9A, act 930 includes generating a patient-level index based on the static claim metric and the plurality of dynamic claim metrics. FIG. 9D further clarifies act 930.

The process of generating the patient-level index is performed by the acts recited in FIG. 9D. For instance, act 930A includes structuring the patient-level index to reflect a set of values that are distributed throughout a time period starting subsequent in time to the transition timestamp. The graph 605 is representative of these values.

Act 930B includes causing the patient-level index to have a starting value reflective of the static claim metric. The patient-level index continues to have the starting value until the first timestamp is reached. For instance, using FIG. 6 and graph 605 as a reference, the starting value of the index in graph 605 is shown as being the static claim metric, which is the value 0.018265.

Act 930C includes reducing, at the first timestamp, the starting value of the index by the first dynamic claim metric. Graph 605 shows how the starting value is reduced at the following timestamp 2023-04-13 because of the two new claims.

Act 930D includes, successively for each new day between the first timestamp and the second timestamp, generating a corresponding new, increased value for the index by dividing the second number of claims by a combination of the second number of days plus a value that reflects a number of days that have elapsed between the first timestamp and said each new day, resulting in the corresponding new, increased value for the index asymptotically increasing towards the starting value. In graph 605, notice how the index values asymptotically increase after an initial drop. The value is asymptotically increasing to reach the original starting value. This increase occurs because the divisor in the earlier equation is increasing with each new passing day while the dividend remains the same. Thus, the result of the division is a progressively decreasing number, and this decreasing number is subtracted from the starting value.

Act 930E includes, at the second timestamp, reducing a latest value of the index by the second dynamic claim metric. In graph 605, the index again drops at the 2023-06-01 timestamp because of the two new claims.

Act 930F includes, successively for each new day subsequent to the second timestamp, generating a different corresponding new, increased value for the index by dividing the third number of claims by a combination of the third number of days plus a second value that reflects a second number of days that have elapsed between the second timestamp and said each new day subsequent to the second timestamp, resulting in the different corresponding new, increased value for the index asymptotically increasing towards the starting value. In graph 605, the index again asymptotically increases after the drop.

Returning to FIG. 9A, act 935 includes aggregating the patient-level index with a plurality of other patient-level indexes associated with the same particular treatment for the medical condition to generate an overall efficacy index for the particular treatment. The overall efficacy index statistically reflects an efficacy of the particular treatment for the medical condition. Graph 605 shows the patient-level index whereas index 500 shows an index that has been formed from the aggregation of multiple different patient-level indexes.

In some implementations, the overall efficacy index for the particular treatment is averaged with a plurality of other overall efficacy indexes for other treatments for the medical condition to generate a medical condition efficacy index. For instance, index 500 of FIG. 5 can be combined with other indexes that have been generated for the medical procedure to create the medical condition efficacy index.

Optionally, an adoption time period for the particular treatment is at least 12 months. In some cases, the second set of timestamps include timestamps that are less than 12 months relative to the transition timestamp and timestamps that are greater than 12 months relative to the transition timestamp.

In some scenarios, the efficacy index is based on the assumption that if the treatment or drug is effective, the average number of daily claims post-transition will likely be smaller than that of pre-transition. Therefore, one aspect of the index that reflects efficacy is the subtraction of the average number of daily claims post-transition from the average number of daily claims pre-transition. It should also be noted how efficacy can also be assessed in economic terms. If the index evaluates the reduction of claims filed before and after the treatment, this reduction can be translated into cost savings, and therefore, into dollars.

The disclosed techniques can be quantified in dollars to show the economical efficacy of the drug. A reduction in claims can be assessed monetarily using the average cost of a claim. This can be done at the patient level because, if one were to add all the patients consuming the drug, the economical efficacy may be affected by external factors such as the popularity of the drug (e.g., the number of patients consuming it). Beneficially, the disclosed embodiments can also identify the most likely condition associated or being treated by the treatment (or drug).

### Efficacy Index Construction—Comparative Analysis: Chemotherapy vs. Radiotherapy

A study was performed to evaluate the efficacy of chemotherapy and radiotherapy on cancer patients by analyzing their healthcare claims before and after the commencement of these therapies. The study selected a cohort of patients undergoing either chemotherapy or radiotherapy, ensuring that all patients had the same type of cancer, thereby standardizing the underlying cause for treatment. The following details will describe the results of that study. It should be noted how these details are provided for example purposes only and should not be viewed as limiting the scope of this disclosure.

The cohort was stratified into two groups based on the therapy received: one group undergoing chemotherapy and the other undergoing radiotherapy. For each patient, the study found the exact date they began their respective therapy.

The study then performed a detailed analysis to compare the average number of healthcare claims (related to the underlying cancer) submitted by patients before starting the therapy against the average number of claims submitted after starting the therapy. This comparison aimed to quantify the impact of each therapy on the frequency of healthcare claims, serving as a proxy for healthcare utilization and potentially, the burden of disease.

For each group, the study calculated the per capita average number of claims both pre-and post-therapy commencement. By averaging these figures across all patients within each therapy group, the study determined the average reduction in healthcare claims attributable to the initiation of chemotherapy or radiotherapy.

This comprehensive analysis allows one to draw conclusions about the relative efficacy of chemotherapy versus radiotherapy in reducing healthcare claims, which may reflect improvements in patient condition or changes in healthcare needs post-therapy:

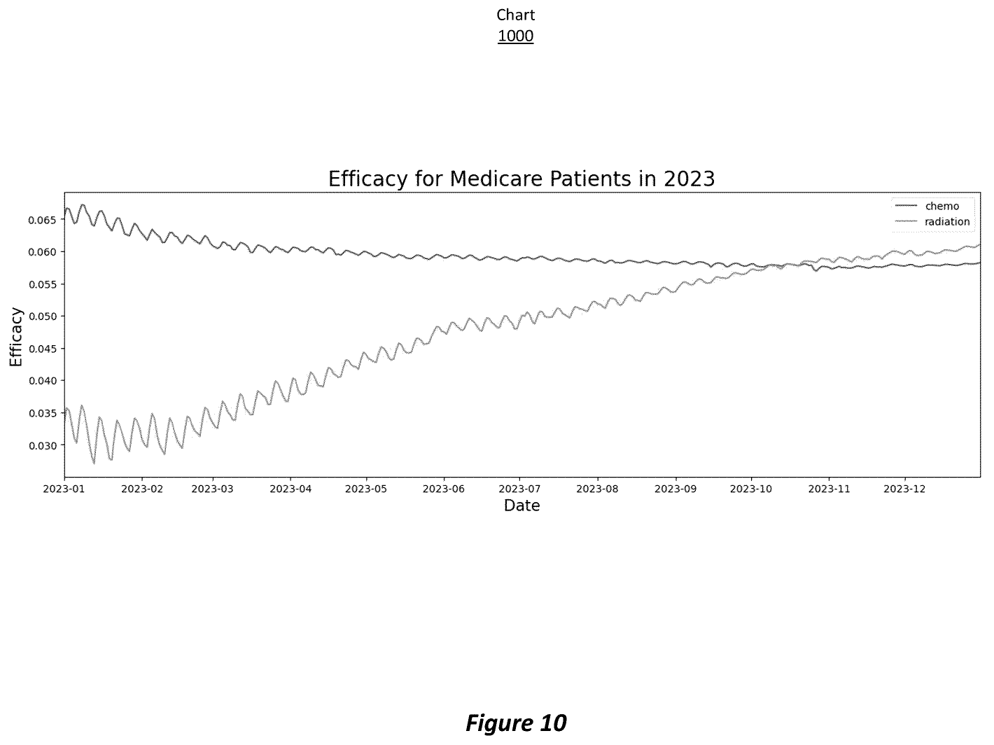

FIG. 10 shows a chart 1000. In chart 1000, the x-axis represents the time lapse during which the study observes the efficacy evolution of both chemotherapy and radiotherapy treatments. The y-axis quantifies efficacy, defined as the average reduction in healthcare claims observed in each patient group on any given day.

At the outset of the study's observation period (index date: Jan. 1, 2023), chemotherapy exhibits an efficacy rate of approximately 0.065 claims per patient. This indicates that, on average, patients who initiated chemotherapy experienced a reduction of 0.065 healthcare claims in the post-therapy period ending on Jan. 1, 2023.

It is worthwhile to note that the duration of the post-therapy period varies depending on the date each patient commenced treatment. Despite this variability, the comparison remains valid because the study used the average daily claims submission rate during the observed period. This rate is a normalized measure, calculated by dividing the total number of claims submitted by the number of days elapsed since the therapy began.

This normalization ensures that the efficacy comparison among individuals is consistent and reliable, regardless of the length of their post-therapy period. By focusing on the average daily claims, the study can accurately assess and compare the impact of chemotherapy and radiotherapy on healthcare claims reduction over time.

Conversely, radiotherapy initially appears less efficacious, with an efficacy rate of 0.035 claims per patient as of Jan. 1, 2023. This suggests that at the start of the observation period, patients undergoing chemotherapy experienced nearly twice the reduction in healthcare claims compared to those undergoing radiotherapy.

However, the efficacy of radiotherapy shows a marked increase over time. While the efficacy of chemotherapy exhibits a slight decline before stabilizing, radiotherapy efficacy begins to rise rapidly. By the period between September and November 2023, radiotherapy's efficacy reaches parity with chemotherapy, achieving an efficacy rate of approximately 0.060 claims per patient.

Notably, as the year progresses, radiotherapy surpasses chemotherapy in efficacy. By the end of the observation period in December 2023, radiotherapy stands out as the most efficacious therapy, demonstrating a higher average reduction in healthcare claims compared to chemotherapy. This dynamic shift highlights the evolving effectiveness of radiotherapy over a prolonged period and underscores its potential advantages in long-term cancer treatment efficacy.

It is worthwhile to note that the index might, in some scenarios, not account for new patients who began their treatment during the observation period (i.e., the year 2023 in this example). In other words, all patients included in both the chemotherapy and radiotherapy groups commenced their respective treatments prior to Jan. 1, 2023, in this example.

This methodological choice ensures that the size of each patient group remains constant throughout the index construction period. By excluding newcomers, this study eliminated potential variability in group sizes, thereby providing a consistent basis for evaluating and comparing the efficacy of the treatments over time. This consistency cane be worthwhile for maintaining the integrity and reliability of the comparative analysis, as newcomers tend to show less efficacy and thus affect negatively the averages:

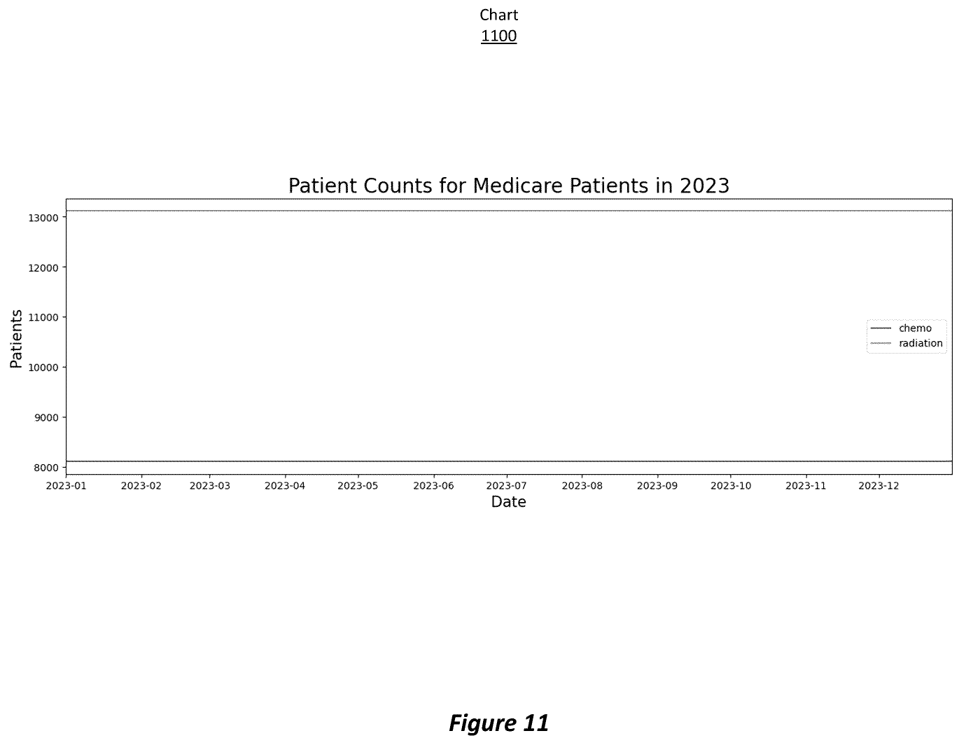

Next, the study evaluated the average daily cost of healthcare claims associated with the treatments during the observation period covered by the index. Although the diagnosis is consistent across both groups, the cost of claims may differ due to the specific treatment being administered. Such a scenario is reflected by chart 1100 in FIG. 11.

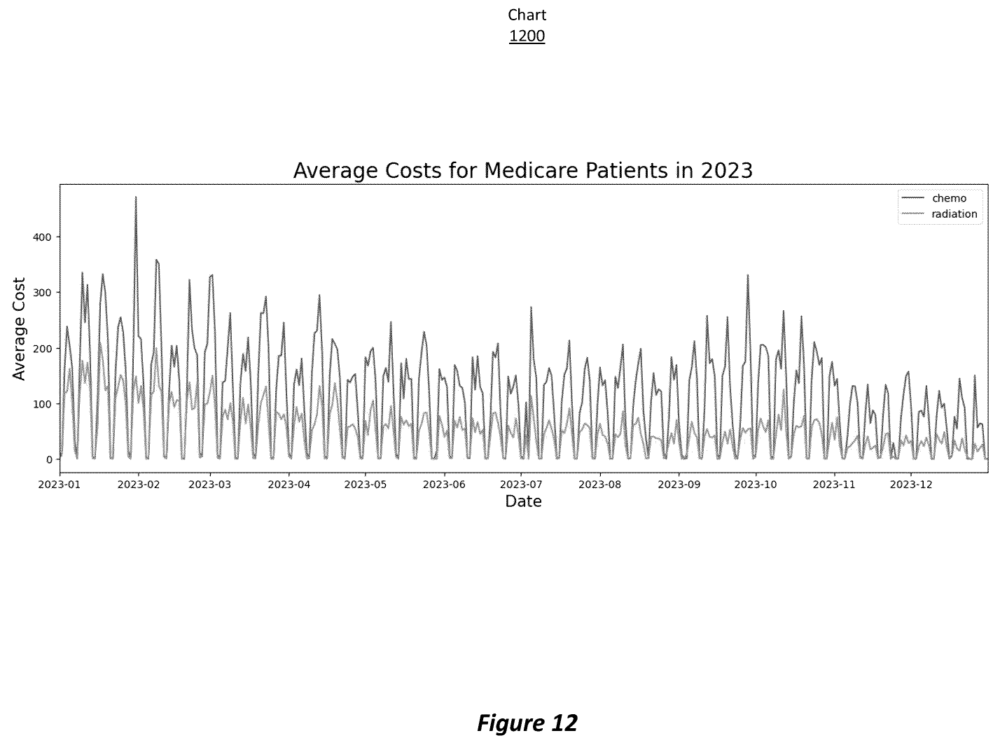

Analyzing the financial data, the study observed variations in the average daily cost of claims between the chemotherapy and radiotherapy groups. Again, these variations are influenced by the distinct nature and requirements of each treatment modality. For this particular example, the study focused on Medicare patients only, but the analysis holds unchangeable for any payer. The average daily cost of claims for both groups is presented in chart 1200 of FIG. 12.

To gain a comprehensive understanding of the financial benefits of each treatment, the study can integrate the efficacy and cost data. Chart 1200 illustrates the average dollar cost per claim, while the efficacy index displays the number of claims saved per patient. By multiplying these two metrics, the study can determine the average cost savings per patient.

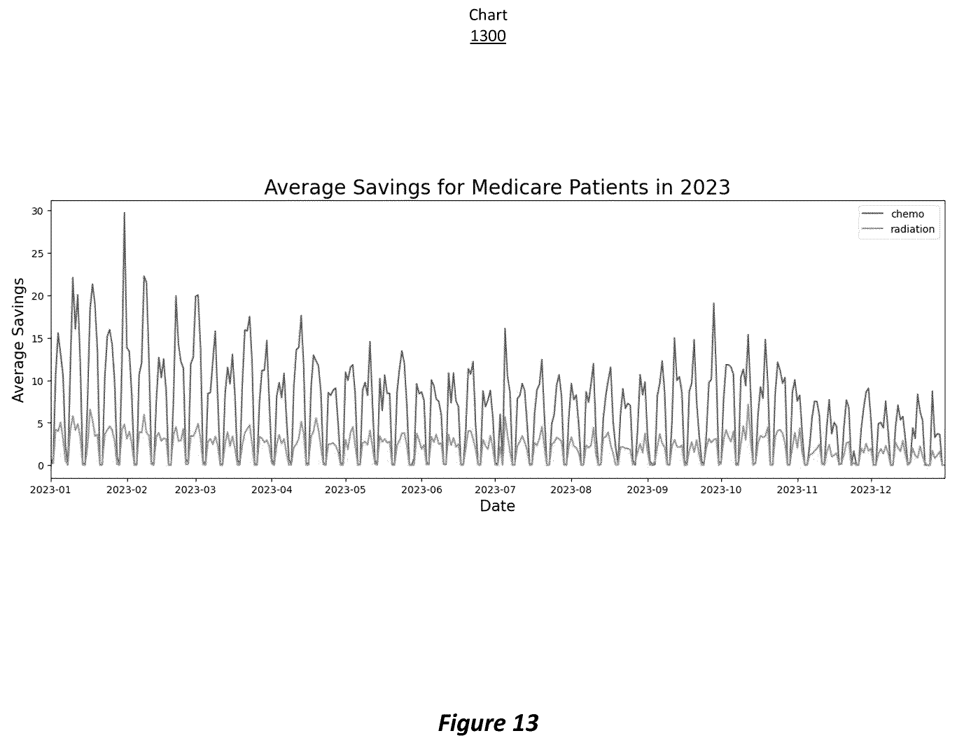

This combined analysis is depicted in chart 1300 of FIG. 13, which presents the average savings per patient resulting from the reduction in healthcare claims due to the treatments. By relating the efficacy of claims reduction to the financial cost per claim, the study obtains a picture of the economic efficiency of chemotherapy and radiotherapy. This integrated view is beneficial for stakeholders to make informed decisions about the cost-effectiveness of cancer treatments.

The final step in this analysis is to create a cumulative chart based on the previously calculated average savings per patient, as shown by chart 1300. This approach allows the study to evaluate the total amount of money saved per patient by the end of the year, depending on the chosen treatment.

To construct this cumulative chart, the study sums the daily savings for each day of the observation period, continuing until the end of the index period (i.e., Dec. 31, 2023). By aggregating the daily savings, the study can visualize the total financial impact of each treatment over the entire year.

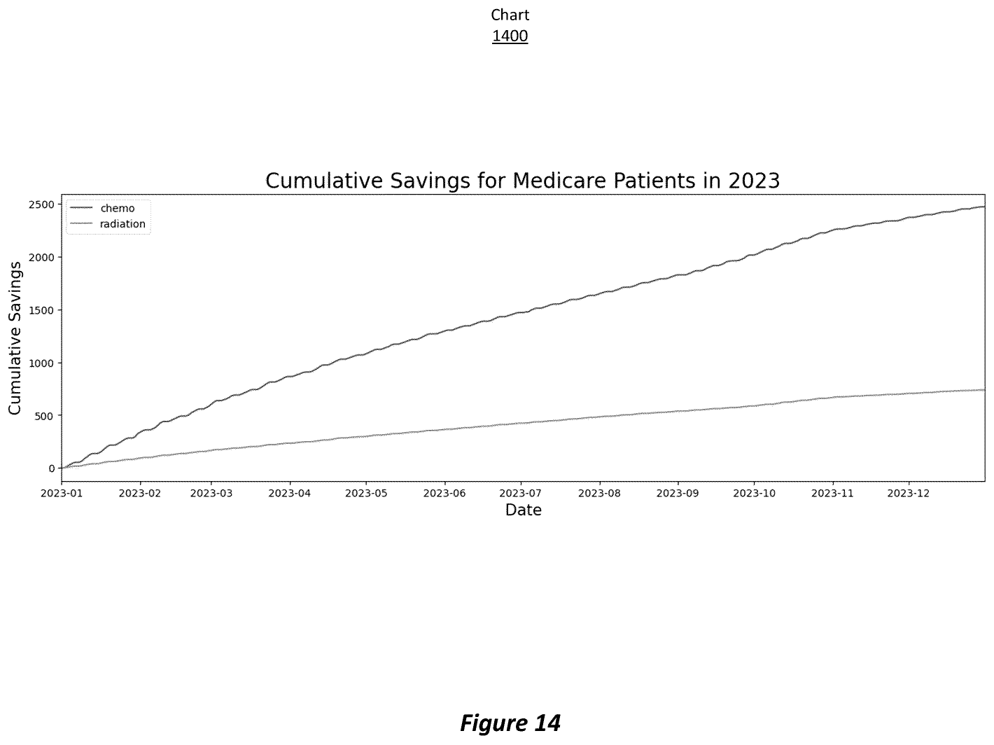

The resulting cumulative savings graphic is presented in chart 1400 of FIG. 14. Chart 1400 provides a depiction of the overall cost savings per patient for chemotherapy and radiotherapy, offering valuable insights into the long-term economic benefits of each treatment option.

From the cumulative analysis over one year, the study observes that patients undergoing chemotherapy save approximately $2,500 per patient, compared to nearly $500 saved by patients undergoing radiotherapy. This suggests that, despite the efficacy index indicating otherwise, chemotherapy is a more cost-effective treatment overall.

This finding highlights a noteworthy consideration: the efficacy of a treatment should not be measured solely by the reduction in healthcare claims. It should also likely account for the economic burden associated with those claims. When the study integrates economic factors into the assessment, the number of claims saved is weighted by their respective costs, leading to a more holistic evaluation of treatment efficacy.

In this case, although radiotherapy shows a rapid increase in claims reduction efficacy, the higher costs associated with these claims result in lower overall savings. Conversely, chemotherapy, despite a slower increase in efficacy, results in greater overall savings due to the lower cost per claim.

Therefore, when considering both the reduction in healthcare claims and the economic burden of those claims, chemotherapy emerges as the more effective treatment option. This comprehensive approach provides a more accurate and meaningful assessment of treatment efficacy, emphasizing the importance of integrating economic considerations into medical evaluations.

Comparative Analysis: Ibrance vs. Verzenio

Another study now aims to demonstrate that the same methodology can be applied to evaluate the efficacy of pharmaceutical drugs. In this section, the study will assess the efficacy of Ibrance and Verzenio, two medications used to treat advanced or metastatic breast cancer. Similar to before, this study is provided for example purposes only and should not be viewed as limiting.

Ibrance (Palbociclib) is a cyclin-dependent kinase (CDK) 4/6 inhibitor that works by inhibiting proteins in cancer cells that help them divide and grow. It is commonly used in combination with hormone therapy to treat HR-positive, HER2-negative breast cancer.

Verzenio (Abemaciclib) is another CDK 4/6 inhibitor, which also targets proteins in cancer cells to prevent their growth and division. Like Ibrance, Verzenio is used for treating HR-positive, HER2-negative advanced or metastatic breast cancer, often in combination with hormone therapy.

By applying the disclosed techniques, the study will analyze the impact of these drugs on healthcare claims and their associated costs. This involves a number of steps, as listed below.

One step is cohort selection, which involves identifying a cohort of patients diagnosed with breast cancer who have been prescribed either Ibrance or Verzenio. Ensuring that all patients commenced their respective treatment before the observation period begins to maintain a constant group size.

Another step involves data collection, which involves recording the number of healthcare claims submitted by each patient before and after starting the treatment. Additionally, documenting the cost of these claims is performed to understand the financial impact.

Another step is efficacy index calculation, which involves calculating the average reduction in healthcare claims per patient for both drugs, similar to the methodology used for chemotherapy and radiotherapy. This will provide a measure of how effective each drug is in reducing healthcare utilization.

Another step involves cost analysis, which involves assessing the average daily cost of claims associated with each drug to understand the economic implications. This step involves analyzing the financial burden of the claims related to the treatments with Ibrance and Verzenio.

Another step involves integration of efficacy and cost data, which includes combining the efficacy index with the cost data to determine the average cost savings per patient. This will provide a comprehensive view of the financial benefits of each drug, allowing the study to see not just how many claims are reduced, but also the associated cost savings.

Another step involves cumulative savings calculation, which includes summing the daily savings over the observation period to create a cumulative savings chart. This chart will highlight the total financial impact of Ibrance and Verzenio by the end of the year, showing the overall cost savings per patient for each drug.

By following this structured approach, the study can effectively compare the efficacy and economic impact of Ibrance and Verzenio, offering valuable insights into their relative cost-effectiveness in treating breast cancer. This methodology ensures a thorough and nuanced evaluation, accounting for both clinical outcomes and financial considerations.

Accordingly, this study aims to evaluate the efficacy of Ibrance and Verzenio on cancer patients by analyzing their healthcare claims before and after the commencement of these therapies. The study selected a cohort of patients undergoing either Ibrance or Verzenio, ensuring that all patients had the same type of cancer, thereby standardizing the underlying cause for treatment.

The cohort was stratified into two groups based on the therapy received: one group undergoing Ibrance and the other undergoing Verzenio. For each patient, the study found the exact date they began their respective therapy.

The study then performed a detailed analysis to compare the average number of healthcare claims (related to the underlying cancer) submitted by patients before starting the therapy against the average number of claims submitted after starting the therapy. This comparison aimed to quantify the impact of each therapy on the frequency of healthcare claims, serving as a proxy for healthcare utilization and potentially, the burden of disease.

For each group, the study calculated the per capita average number of claims both pre-and post-therapy commencement. By averaging these figures across all patients within each therapy group, the study determined the average reduction in healthcare claims attributable to the initiation of Ibrance or Verzenio.

This comprehensive analysis allows the study to draw conclusions about the relative efficacy of Ibrance versus Verzenio in reducing healthcare claims, which may reflect improvements in patient condition or changes in healthcare needs post-therapy.

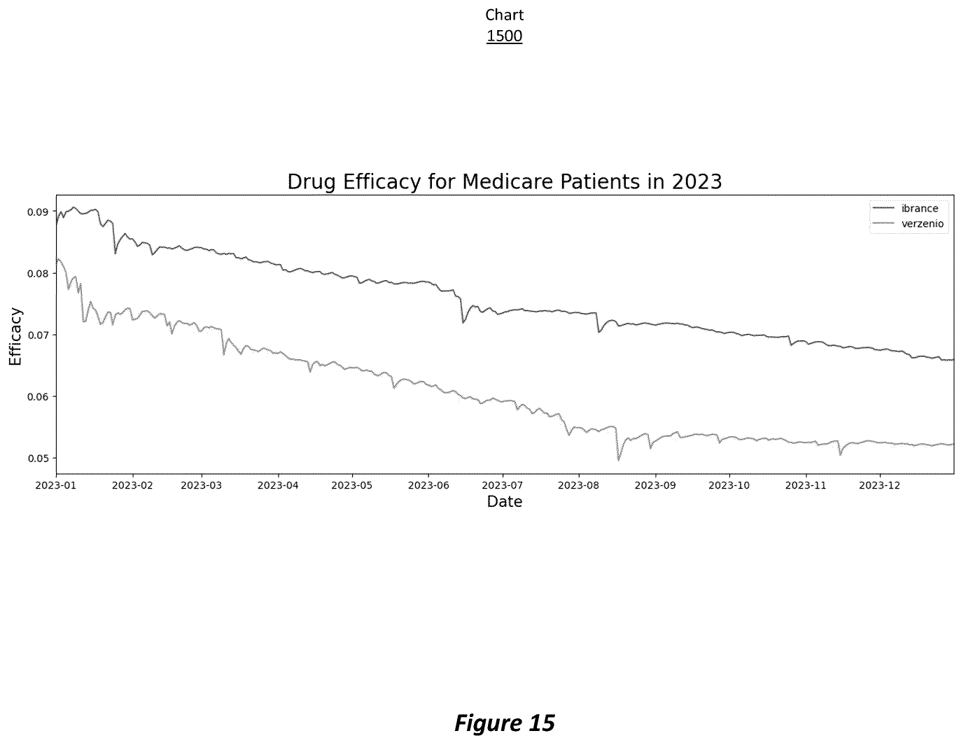

In chart 1500 of FIG. 15, the x-axis represents the time lapse during which the study observes the efficacy evolution of both Ibrance and Verzenio drugs. The y-axis quantifies efficacy, defined as the average reduction in healthcare claims observed in each patient group on any given day.

At the outset of the observation period (index date: Jan. 1, 2023), Ibrance exhibits an efficacy rate of approximately 0.09 claims per patient. This indicates that, on average, patients who initiated Ibrance experienced a reduction of 0.09 healthcare claims in the post-therapy period ending on Jan. 1, 2023.

It is worthwhile to note that the duration of the post-therapy period varies depending on the date each patient commenced treatment. Despite this variability, the comparison remains valid because the study uses the average daily claims submission rate during the observed period. This rate is a normalized measure, calculated by dividing the total number of claims submitted by the number of days elapsed since the drug-based therapy began.

This normalization ensures that the efficacy comparison among individuals is consistent and reliable, regardless of the length of their post-therapy period. By focusing on the average daily claims, the study can accurately assess and compare the impact of Ibrance and Verzenio on healthcare claims reduction over time.

Conversely, Verzenio initially appears less efficacious, with an efficacy rate of 0.083 claims per patient as of Jan. 1, 2023. This suggests that at the start of the observation period, patients taking Ibrance experienced nearly 0.007 of reduction in healthcare claims compared to those undergoing Verzenio. This trend is kept across the year, ending with a slightly wider difference at the end.

It is worthwhile to note that this implementation of the index does not account for new patients who began their treatment during the observation period (i.e., the year 2023 in this example). In other words, all patients included in both the Ibrance and Verzenio groups commenced their respective treatments prior to Jan. 1, 2023.

This methodological choice ensures that the size of each patient group remains constant throughout the index construction period. By excluding newcomers, the study eliminates potential variability in group sizes, thereby providing a consistent basis for evaluating and comparing the efficacy of the treatments over time. This consistency is fundamental for maintaining the integrity and reliability of the comparative analysis, as newcomers tend to show less efficacy and thus affect the averages negatively.

Next, the study evaluated the average daily cost of healthcare claims associated with the treatments during the observation period covered by the index. Although the diagnosis is consistent across both groups, the cost of claims may differ due to the specific treatment being administered.

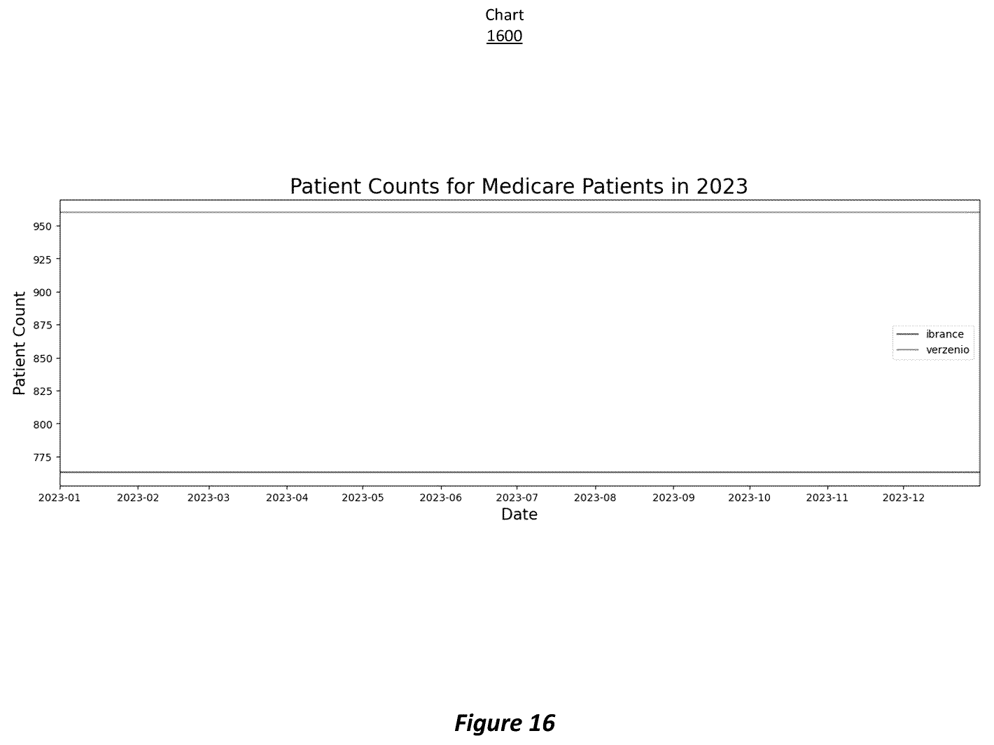

Analyzing the financial data, the study observes variations in the average daily cost of claims between the Ibrance and Verzenio groups. Again, these variations are influenced by the distinct nature and requirements of each treatment modality. For this particular example, the study focused on Medicare patients only, but the analysis holds unchangeable for any payer. The average daily cost of claims for both groups is presented in chart 1600 of FIG. 16.

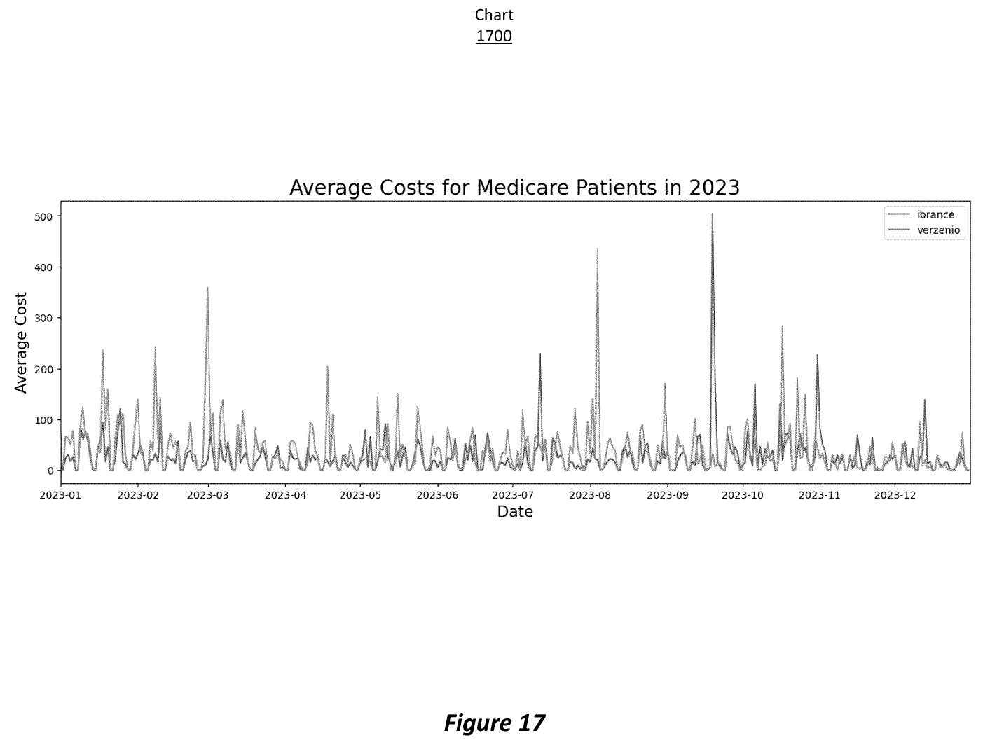

To gain a comprehensive understanding of the financial benefits of each treatment, the study can integrate the efficacy and cost data. Chart 1700 of FIG. 17 illustrates the average dollar cost per claim, while the efficacy index displays the number of claims saved per patient. By multiplying these two metrics, the study can determine the average cost savings per patient.

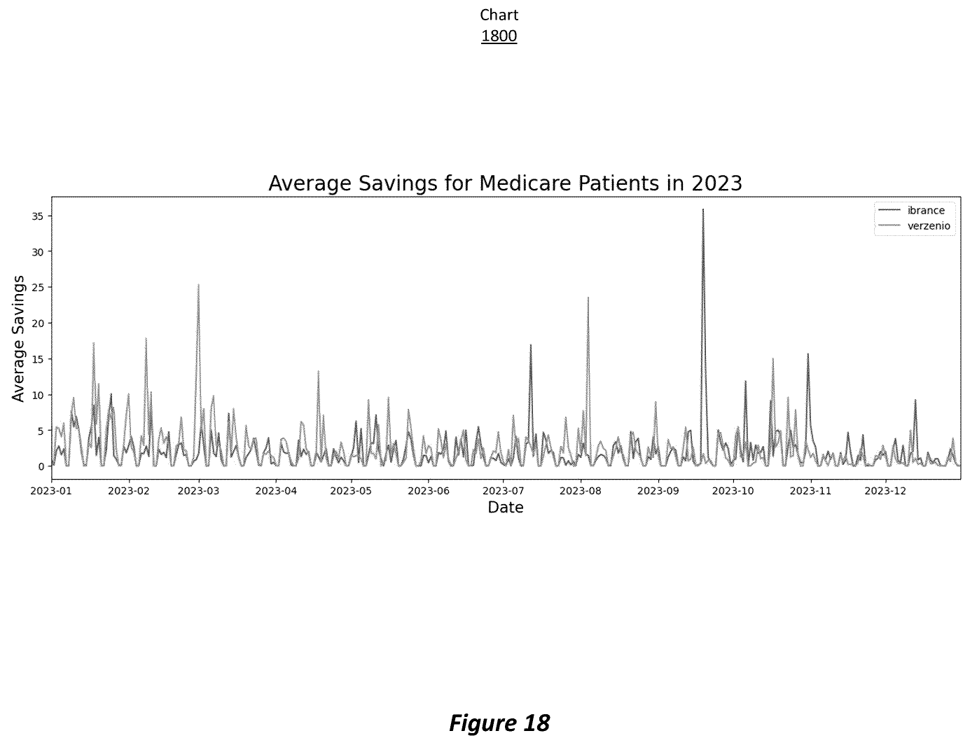

This combined analysis is depicted in chart 1800 of FIG. 18, which presents the average savings per patient resulting from the reduction in healthcare claims due to the treatments. By relating the efficacy of claims reduction to the financial cost per claim, the study obtains a clear picture of the economic efficiency of Ibrance and Verzenio. This integrated view is beneficial for stakeholders to make informed decisions about the cost-effectiveness of cancer drugs.

The final step in this analysis is to create a cumulative chart based on the previously calculated average savings per patient. This approach allows the study to evaluate the total amount of money saved per patient by the end of the year, depending on the chosen treatment.

To construct this cumulative chart, the study sums the daily savings for each day of the observation period, continuing until the end of the index period (i.e., Dec. 31, 2023). By aggregating the daily savings, the study can visualize the total financial impact of each treatment over the entire year.

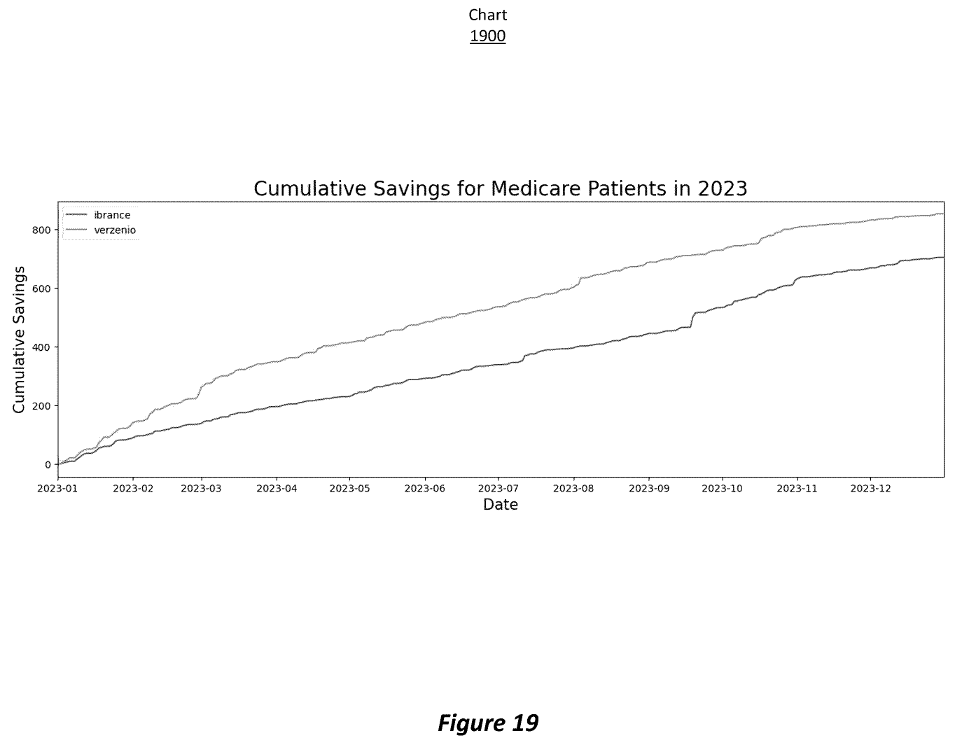

The resulting cumulative savings graphic is presented in chart 1900 of FIG. 19. Chart 1900 provides a depiction of the overall cost savings per patient for Ibrance and Verzenio, offering valuable insights into the long-term economic benefits of each drug option.

From the cumulative analysis over one year, the study observes that patients undergoing Ibrance save approximately $680 per patient, compared to nearly $800 saved by patients undergoing Verzenio. This suggests that, despite the efficacy index indicating otherwise, Verizenio is a more cost-effective treatment overall.

This example, like the previous one, also highlights a notable consideration: the efficacy of a treatment should not be measured solely by the reduction in healthcare claims. It must also account for the economic burden associated with those claims. When the study integrates economic factors into the assessment, the number of claims saved is weighted by their respective costs, leading to a more holistic evaluation of treatment efficacy.

In this case, although Ibrance performed better in claims reduction efficacy, the higher costs associated with these claims result in lower overall savings. Conversely, Verzenio, despite a worse behavior in efficacy, results in greater overall savings due to the lower cost per claim.

Therefore, when considering both the reduction in healthcare claims and the economic burden of those claims, Verzenio emerges as the more effective treatment option. This comprehensive approach provides a more accurate and meaningful assessment of treatment efficacy, emphasizing the importance of integrating economic considerations into medical evaluations.

### Considerations

The cumulative graphs are based on a per-patient analysis and remain so to ensure accuracy. If the study were to aggregate the savings for all patients by multiplying the per capita savings by the total number of patients, it would introduce bias into the analysis. This is because the number of patients in each treatment group differs, as illustrated in the charts showing the group sizes.

This discrepancy arises for various reasons, such as the popularity or market longevity of a drug/therapy (e.g., drug adoption rate). In the analysis, the study addresses this by averaging the results per patient. Adding up all the savings linearly based on the number of patients would unfairly skew the results.

Therefore, the study maintains the per capita measure to ensure that the efficacy analysis is independent of group size. This approach focuses on the average savings per patient, providing a fair comparison between treatments. However, multiplying the per capita metric by the number of patients can be a valid approach when isolating the efficacy of a drug within a larger population (i.e., without comparing to other drugs/therapies). This method offers insights into the overall impact of a treatment across the entire patient cohort, but for this specific efficacy analysis, the study concentrates on the per capita measure to maintain consistency and fairness.

Now, it is possible to conduct a series of tests to analyze the impact of efficacy on final average savings in comparison to average costs. This step is beneficial because initial observations from the previous examples suggest that savings might depend more heavily on the average cost of the claims than on any other factor. Consequently, treatments or therapies with higher efficacy in terms of claim reduction could appear less effective when the cost of claims is taken into account.

To explore this further, the study will examine various scenarios to determine how changes in efficacy and cost interact to affect overall savings. By systematically varying these factors, the study can assess the relative importance of each in determining the final economic benefits of a treatment.

The claim here is that efficacy does not have as significant an effect on final average savings compared to average costs. The study observes that the average savings graph for chemotherapy remains consistent between average costs and average savings. This consistency arises because the efficacy of chemotherapy changes very little over time, essentially remaining constant. Consequently, efficacy has a minimal impact on the shape of the final savings curve for chemotherapy.

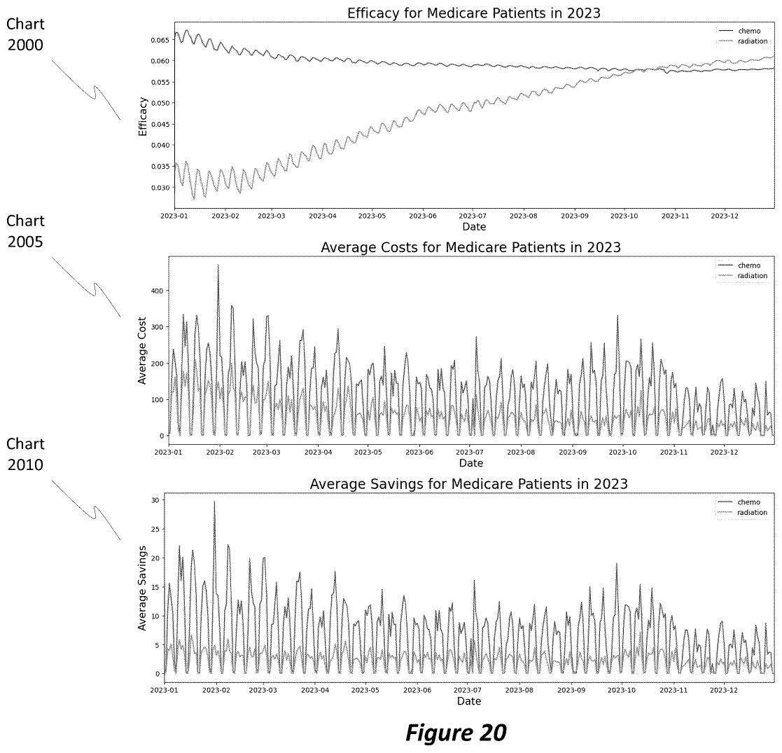

In contrast, efficacy does influence the savings for radiotherapy. In the average savings graph, the local maximums are approximately the same value, indicating a different pattern compared to the average costs graph, which shows a decreasing trend, and the efficacy graph, which shows an increasing trend. These opposing trends cancel each other out, resulting in the constant trend observed in average savings for radiotherapy. Charts 2000, 2005, and 2010 in FIG. 20 are illustrative.

By swapping the efficacy of chemotherapy and radiotherapy, the study can observe that efficacy does indeed have an impact. Although chemotherapy consistently incurs higher average costs throughout the year, lower efficacy ratings at the beginning of the year cause its average savings to drop significantly, even falling below those of radiotherapy. While it might seem that this difference diminishes later in the year, this is because chemotherapy's efficacy increases to nearly match that of radiotherapy in this example. As efficacy becomes a less significant factor in distinguishing between the two treatments, average costs emerge as the most relevant factor.

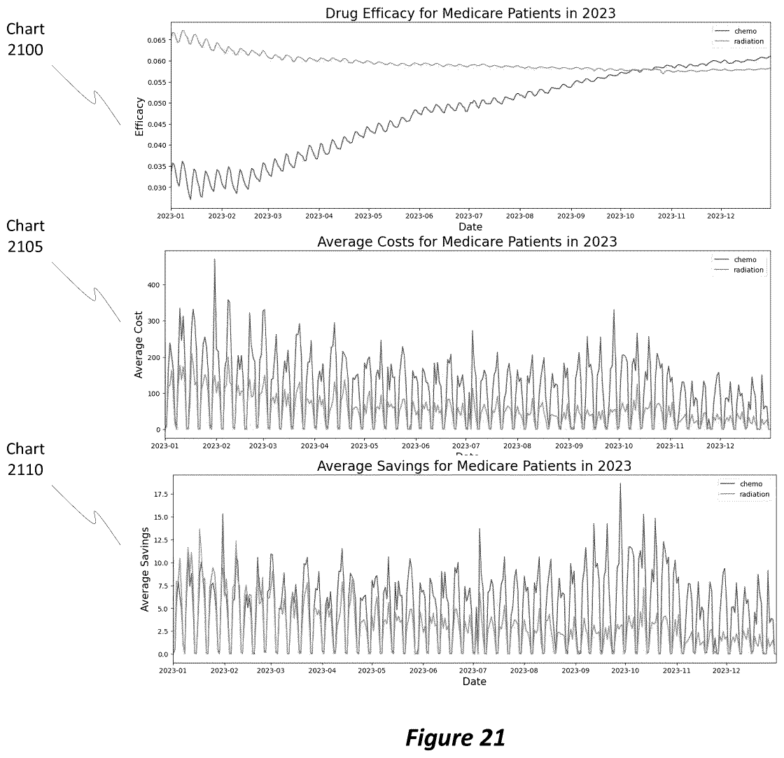

Returning to the original point, chemotherapy is clearly more expensive per patient. When efficacy is equalized, patients ultimately save more money with chemotherapy due to its higher initial cost and the subsequent cost savings realized through increased efficacy. Charts 2100, 2105, and 2110 of FIG. 21 are illustrative.

At first glance, chemotherapy appears to be approximately twice as expensive per patient throughout 2023 compared to radiotherapy. What if the study made the efficacy of chemotherapy half that of radiotherapy?

This change significantly impacts the average savings comparison between chemotherapy and radiotherapy, with the lines in the graph almost overlapping. Why does this happen? Because both efficacy and cost are equally important metrics. If you have half the efficacy but double the cost, the study can expect the same amount of savings.

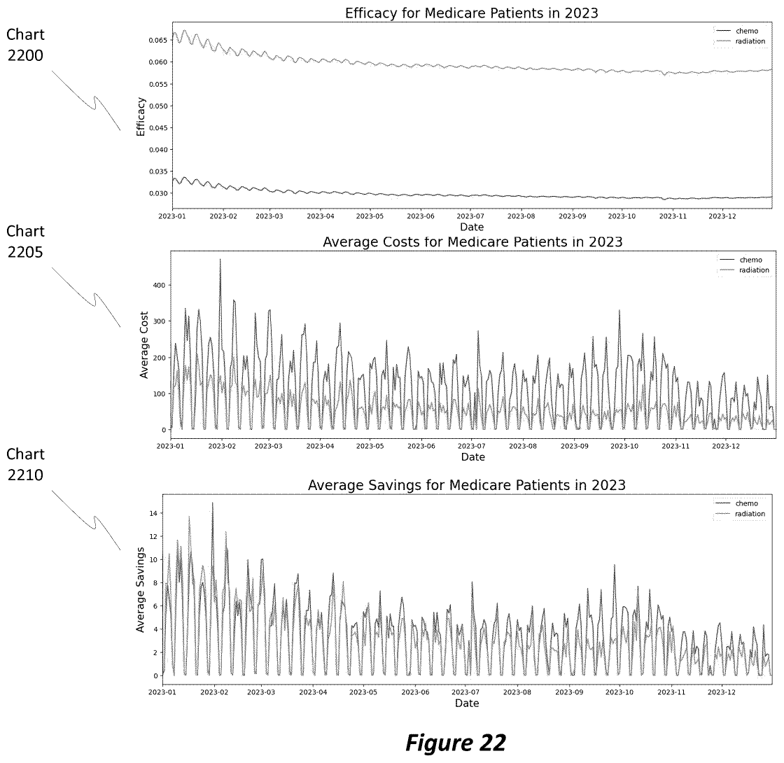

In simpler terms, “Yes, your procedure may cost twice as much, but if it only works half as often, why not choose the half-priced procedure that is twice as effective?” This highlights the balance between cost and efficacy in determining the overall savings for patients. Charts 2200, 2205, and 2210 of FIG. 22 are illustrative.

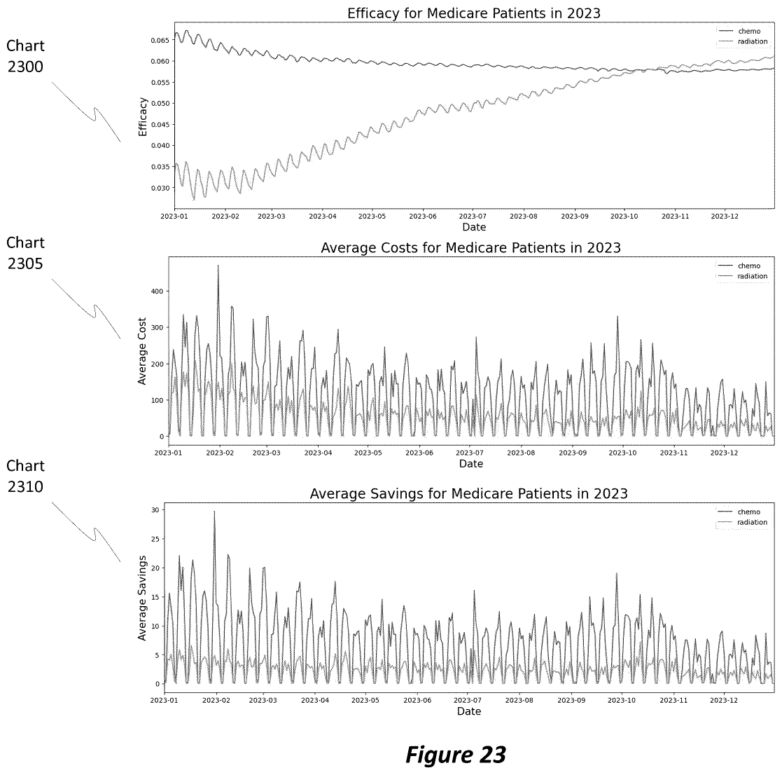

The reason why it seems like efficacy does not appear to have much of an impact on average savings comes down to a few things. Chemotherapy's efficacy is close to constant, so its effect on the average savings is also constant. Efficacy for chemotherapy and average costs for chemotherapy are both greater than radiation's. So, it makes sense that chemotherapy is going to be significantly greater in average savings as well. When comparing two drugs/procedures/etc., the raw values in efficacy and average costs are less important than the multiplicative differences between the two. The latter gives a good indication of comparing in the final average savings calculation. FIGS. 2300, 2305, and 2310 of FIG. 23 are illustrative.

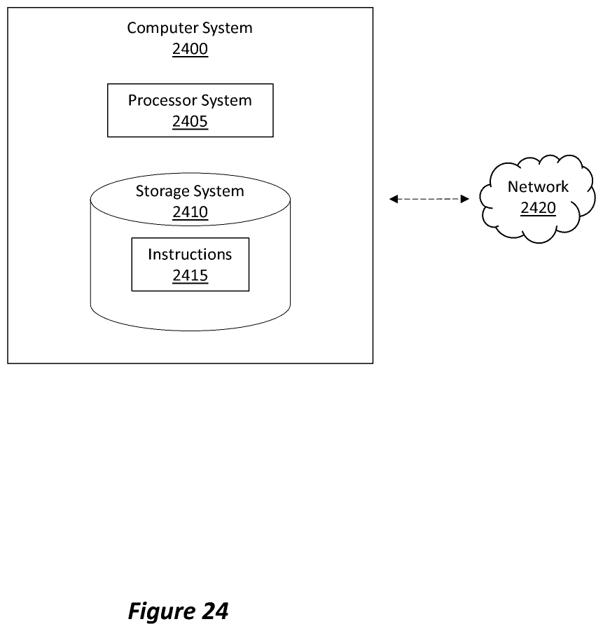

Attention will now be directed to FIG. 24 which illustrates an example computer system 2400 that may include and/or be used to perform any of the operations described herein. For instance, computer system 2400 can implement service 105 of FIG. 1.

Computer system 2400 may take various different forms. For example, computer system 2400 may be embodied as a tablet, a desktop, a laptop, a mobile device, or a standalone device, such as those described throughout this disclosure. Computer system 2400 may also be a distributed system that includes one or more connected computing components/devices that are in communication with computer system 2400.

In its most basic configuration, computer system 2400 includes various different components. FIG. 24 shows that computer system 2400 includes a processor system 2405 that includes one or more processor(s) (aka a “hardware processing unit”) and a storage system 2410.

Regarding the processor(s) of the processor system 2405, it will be appreciated that the functionality described herein can be performed, at least in part, by one or more hardware logic components (e.g., the processor(s)). For example, and without limitation, illustrative types of hardware logic components/processors that can be used include Field-Programmable Gate Arrays (“FPGA”), Program-Specific or Application-Specific Integrated Circuits (“ASIC”), Program-Specific Standard Products (“ASSP”), System-On-A-Chip Systems (“SOC”), Complex Programmable Logic Devices (“CPLD”), Central Processing Units (“CPU”), Graphical Processing Units (“GPU”), or any other type of programmable hardware.

As used herein, the terms “executable module,” “executable component,” “component,” “module,” “service,” or “engine” can refer to hardware processing units or to software objects, routines, or methods that may be executed on computer system 2400. The different components, modules, engines, and services described herein may be implemented as objects or processors that execute on computer system 2400 (e.g. as separate threads).

Storage system 2410 may be physical system memory, which may be volatile, non-volatile, or some combination of the two. The term “memory” may also be used herein to refer to non-volatile mass storage such as physical storage media. If computer system 2400 is distributed, the processing, memory, and/or storage capability may be distributed as well.

Storage system 2410 is shown as including executable instructions 2415. The executable instructions 2415 represent instructions that are executable by the processor(s) the processor system 2405 to perform the disclosed operations, such as those described in the various methods.

The disclosed embodiments may comprise or utilize a special-purpose or general-purpose computer including computer hardware, such as, for example, one or more processors and system memory, as discussed in greater detail below. Embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are “physical computer storage media” or a “hardware storage device.” Furthermore, computer-readable storage media, which includes physical computer storage media and hardware storage devices, exclude signals, carrier waves, and propagating signals. On the other hand, computer-readable media that carry computer-executable instructions are “transmission media” and include signals, carrier waves, and propagating signals. Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in the form of computer-executable instructions, data, or data structures and that can be accessed by a general-purpose or special-purpose computer.

Computer system 2400 may also be connected (via a wired or wireless connection) to external sensors (e.g., one or more remote cameras) or devices via a network 2420. For example, computer system 2400 can communicate with any number devices or cloud services to obtain or process data. In some cases, network 2420 may itself be a cloud network. Furthermore, computer system 2400 may also be connected through one or more wired or wireless networks to remote/separate computer systems(s) that are configured to perform any of the processing described with regard to computer system 2400.

A “network,” like network 2420, is defined as one or more data links and/or data switches that enable the transport of electronic data between computer systems, modules, and/or other electronic devices. When information is transferred, or provided, over a network (either hardwired, wireless, or a combination of hardwired and wireless) to a computer, the computer properly views the connection as a transmission medium. Computer system 2400 will include one or more communication channels that are used to communicate with the network 2420. Transmissions media include a network that can be used to carry data or desired program code means in the form of computer-executable instructions or in the form of data structures. Further, these computer-executable instructions can be accessed by a general-purpose or special-purpose computer. Combinations of the above should also be included within the scope of computer-readable media.

Upon reaching various computer system components, program code means in the form of computer-executable instructions or data structures can be transferred automatically from transmission media to computer storage media (or vice versa). For example, computer-executable instructions or data structures received over a network or data link can be buffered in RAM within a network interface module (e.g., a network interface card or “NIC”) and then eventually transferred to computer system RAM and/or to less volatile computer storage media at a computer system. Thus, it should be understood that computer storage media can be included in computer system components that also (or even primarily) utilize transmission media.

Computer-executable (or computer-interpretable) instructions comprise, for example, instructions that cause a general-purpose computer, special-purpose computer, or special-purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the embodiments may be practiced in network computing environments with many types of computer system configurations, including personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The embodiments may also be practiced in distributed system environments where local and remote computer systems that are linked (either by hardwired data links, wireless data links, or by a combination of hardwired and wireless data links) through a network each perform tasks (e.g. cloud computing, cloud services and the like). In a distributed system environment, program modules may be located in both local and remote memory storage devices.

The present invention may be embodied in other specific forms without departing from its characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for generating an overall efficacy index that statistically reflects an efficacy of a particular treatment used to treat a medical condition, said method comprising:
identifying a transition timestamp reflective of when a patient begins a particular treatment for a medical condition of the patient;
accessing a first set of claims associated with the patient, the first set of claims being claims related to the patient's medical condition prior to the transition timestamp, wherein each claim in the first set of claims is associated with a corresponding timestamp, a combination of which constitutes a first set of timestamps, and wherein the first set of timestamps all pre-date the transition timestamp;
determining a static claim metric for the patient's medical condition using (i) a total number of claims included in the first set of claims and (ii) a number of days spanning between an earliest timestamp included among the first set of timestamps and the transition timestamp;
accessing a second set of claims associated with the patient's medical condition, the second set of claims being claims related to the particular treatment the patient performed to address the patient's medical condition, wherein each claim in the second set of claims is associated with a corresponding timestamp, a combination of which constitutes a second set of timestamps, and wherein the second set of timestamps all post-date the transition timestamp;
determining a plurality of dynamic claim metrics for the patient's medical condition using (i) the second set of claims, (ii) the second set of timestamps, and (iii) the transition timestamp;
generating a patient-level index based on the static claim metric and the plurality of dynamic claim metrics; and
aggregating the patient-level index with a plurality of other patient-level indexes associated with the same particular treatment for the medical condition to generate an overall efficacy index for the particular treatment, the overall efficacy index statistically reflecting an efficacy of the particular treatment for the medical condition.

2. The method of claim 1, wherein determining the static claim metric is performed by:
determining the number of days spanning between the earliest timestamp included among the first set of timestamps and the transition timestamp;
determining the total number of claims included in the first set of claims; and
dividing the total number of claims by the number of days to generate a fixed average daily claim value, wherein the fixed average daily claim value constitutes the static claim metric.

3. The method of claim 2, wherein determining the plurality of dynamic claim metrics is performed by:
using the second set of claims to form different groups of claims that are grouped based on the second set of timestamps, wherein the groups of claims include at least a first group of claims associated with a first timestamp and a second group of claims associated with a second timestamp, the second timestamp being after the first timestamp;
for the first group of claims:
determining a second number of days spanning between the transition timestamp and the first timestamp;
determining a second number of claims included in the first group;
computing a first dynamic claim metric by dividing the second number of claims by the second number of days;
for the second group of claims:
determining a third number of days spanning between the transition timestamp and the second timestamp;
determining a third number of claims included in the second group;
computing a second dynamic claim metric by dividing the third number of claims by the third number of days, the second dynamic claim metric not being a fixed metric and is updatable at a periodic rate.

4. The method of claim 3, wherein generating the patient-level index is performed by:
structuring the patient-level index to reflect a set of values that are distributed throughout a time period starting subsequent in time to the transition timestamp;
causing the patient-level index to have a starting value reflective of the static claim metric, wherein the patient-level index continues to have said starting value until the first timestamp is reached;
at the first timestamp, reducing the starting value of the index by the first dynamic claim metric;
successively for each new day between the first timestamp and the second timestamp, generating a corresponding new, increased value for the index by dividing the second number of claims by a combination of the second number of days plus a value that reflects a number of days that have elapsed between the first timestamp and said each new day, resulting in the corresponding new, increased value for the index asymptotically increasing towards the starting value;
at the second timestamp, reducing a latest value of the index by the second dynamic claim metric; and
successively for each new day subsequent to the second timestamp, generating a different corresponding new, increased value for the index by dividing the third number of claims by a combination of the third number of days plus a second value that reflects a second number of days that have elapsed between the second timestamp and said each new day subsequent to the second timestamp, resulting in the different corresponding new, increased value for the index asymptotically increasing towards the starting value.

5. The method of claim 1, wherein the treatment involves a pharmaceutical such that the index reflects an efficacy of the pharmaceutical.

6. The method of claim 1, wherein the treatment involves a medical procedure such that the index reflects an efficacy of the medical procedure.

7. The method of claim 1, wherein the overall efficacy index for the particular treatment is averaged with a plurality of other overall efficacy indexes for other treatments for the medical condition to generate a medical condition efficacy index.

8. The method of claim 1, wherein an adoption time period for the particular treatment is at least 12 months, and wherein the second set of timestamps include timestamps that are less than 12 months relative to the transition timestamp and timestamps that are greater than 12 months relative to the transition timestamp.

9. A computer system that generates an overall efficacy index that statistically reflects an efficacy of a particular treatment used to treat a medical condition, said computer system comprising:
one or more processors; and
one or more hardware storage devices that store instructions that are executable by the one or more processors to cause the computer system to:
identify a transition timestamp reflective of when a patient begins a particular treatment for a medical condition of the patient;
access a first set of claims associated with the patient, the first set of claims being claims related to the patient's medical condition prior to the transition timestamp, wherein each claim in the first set of claims is associated with a corresponding timestamp, a combination of which constitutes a first set of timestamps, and wherein the first set of timestamps all pre-date the transition timestamp;
determine a static claim metric for the patient's medical condition using (i) a total number of claims included in the first set of claims and (ii) a number of days spanning between an earliest timestamp included among the first set of timestamps and the transition timestamp;
access a second set of claims associated with the patient's medical condition, the second set of claims being claims related to the particular treatment the patient performed to address the patient's medical condition, wherein each claim in the second set of claims is associated with a corresponding timestamp, a combination of which constitutes a second set of timestamps, and wherein the second set of timestamps all post-date the transition timestamp;
determine a plurality of dynamic claim metrics for the patient's medical condition using (i) the second set of claims, (ii) the second set of timestamps, and (iii) the transition timestamp;
generate a patient-level index based on the static claim metric and the plurality of dynamic claim metrics; and
aggregate the patient-level index with a plurality of other patient-level indexes associated with the same particular treatment for the medical condition to generate an overall efficacy index for the particular treatment, the overall efficacy index statistically reflecting an efficacy of the particular treatment for the medical condition.

10. The computer system of claim 9, wherein determining the static claim metric is performed by:
determining the number of days spanning between the earliest timestamp included among the first set of timestamps and the transition timestamp;
determining the total number of claims included in the first set of claims; and
dividing the total number of claims by the number of days to generate a fixed average daily claim value, wherein the fixed average daily claim value constitutes the static claim metric.

11. The computer system of claim 10, wherein determining the plurality of dynamic claim metrics is performed by:
using the second set of claims to form different groups of claims that are grouped based on the second set of timestamps, wherein the groups of claims include at least a first group of claims associated with a first timestamp and a second group of claims associated with a second timestamp, the second timestamp being after the first timestamp;
for the first group of claims:
determining a second number of days spanning between the transition timestamp and the first timestamp;
determining a second number of claims included in the first group;
computing a first dynamic claim metric by dividing the second number of claims by the second number of days;
for the second group of claims:
determining a third number of days spanning between the transition timestamp and the second timestamp;
determining a third number of claims included in the second group;
computing a second dynamic claim metric by dividing the third number of claims by the third number of days.

12. The computer system of claim 11, wherein generating the patient-level index is performed by:
structuring the patient-level index to reflect a set of values that are distributed throughout a time period starting subsequent in time to the transition timestamp;
causing the patient-level index to have a starting value reflective of the static claim metric, wherein the patient-level index continues to have said starting value until the first timestamp is reached;
at the first timestamp, reducing the starting value of the index by the first dynamic claim metric;
successively for each new day between the first timestamp and the second timestamp, generating a corresponding new, increased value for the index by dividing the second number of claims by a combination of the second number of days plus a value that reflects a number of days that have elapsed between the first timestamp and said each new day, resulting in the corresponding new, increased value for the index asymptotically increasing towards the starting value;
at the second timestamp, reducing a latest value of the index by the second dynamic claim metric; and
successively for each new day subsequent to the second timestamp, generating a different corresponding new, increased value for the index by dividing the third number of claims by a combination of the third number of days plus a second value that reflects a second number of days that have elapsed between the second timestamp and said each new day subsequent to the second timestamp, resulting in the different corresponding new, increased value for the index asymptotically increasing towards the starting value.

13. The computer system of claim 9, wherein the treatment involves a pharmaceutical such that the index reflects an efficacy of the pharmaceutical.

14. The computer system of claim 9, wherein the treatment involves a medical procedure such that the index reflects an efficacy of the medical procedure.

15. The computer system of claim 9, wherein the overall efficacy index for the particular treatment is averaged with a plurality of other overall efficacy indexes for other treatments for the medical condition to generate a medical condition efficacy index.

16. The computer system of claim 9, wherein an adoption time period for the particular treatment is at least 12 months, and wherein the second set of timestamps include timestamps that are less than 12 months relative to the transition timestamp and timestamps that are greater than 12 months relative to the transition timestamp.

17. One or more hardware storage devices that store instructions that are executable by one or more processors to cause the one or more processors to:
identify a transition timestamp reflective of when a patient begins a particular treatment for a medical condition of the patient;
access a first set of claims associated with the patient, the first set of claims being claims related to the patient's medical condition prior to the transition timestamp, wherein each claim in the first set of claims is associated with a corresponding timestamp, a combination of which constitutes a first set of timestamps, and wherein the first set of timestamps all pre-date the transition timestamp;
determine a static claim metric for the patient's medical condition using (i) a total number of claims included in the first set of claims and (ii) a number of days spanning between an earliest timestamp included among the first set of timestamps and the transition timestamp;
access a second set of claims associated with the patient's medical condition, the second set of claims being claims related to the particular treatment the patient performed to address the patient's medical condition, wherein each claim in the second set of claims is associated with a corresponding timestamp, a combination of which constitutes a second set of timestamps, and wherein the second set of timestamps all post-date the transition timestamp;
determine a plurality of dynamic claim metrics for the patient's medical condition using (i) the second set of claims, (ii) the second set of timestamps, and (iii) the transition timestamp;
generate a patient-level index based on the static claim metric and the plurality of dynamic claim metrics; and
aggregate the patient-level index with a plurality of other patient-level indexes associated with the same particular treatment for the medical condition to generate an overall efficacy index for the particular treatment, the overall efficacy index statistically reflecting an efficacy of the particular treatment for the medical condition.

18. The one or more hardware storage devices of claim 17, wherein determining the static claim metric is performed by:
determining the number of days spanning between the earliest timestamp included among the first set of timestamps and the transition timestamp;
determining the total number of claims included in the first set of claims; and
dividing the total number of claims by the number of days to generate a fixed average daily claim value, wherein the fixed average daily claim value constitutes the static claim metric.

19. The one or more hardware storage devices of claim 18, wherein determining the plurality of dynamic claim metrics is performed by:
using the second set of claims to form different groups of claims that are grouped based on the second set of timestamps, wherein the groups of claims include at least a first group of claims associated with a first timestamp and a second group of claims associated with a second timestamp, the second timestamp being after the first timestamp;
for the first group of claims:
determining a second number of days spanning between the transition timestamp and the first timestamp;
determining a second number of claims included in the first group;
computing a first dynamic claim metric by dividing the second number of claims by the second number of days;
for the second group of claims:
determining a third number of days spanning between the transition timestamp and the second timestamp;
determining a third number of claims included in the second group;
computing a second dynamic claim metric by dividing the third number of claims by the third number of days.

20. The one or more hardware storage devices of claim 19, wherein generating the patient-level index is performed by:
structuring the patient-level index to reflect a set of values that are distributed throughout a time period starting subsequent in time to the transition timestamp;
causing the patient-level index to have a starting value reflective of the static claim metric, wherein the patient-level index continues to have said starting value until the first timestamp is reached;
at the first timestamp, reducing the starting value of the index by the first dynamic claim metric;
successively for each new day between the first timestamp and the second timestamp, generating a corresponding new, increased value for the index by dividing the second number of claims by a combination of the second number of days plus a value that reflects a number of days that have elapsed between the first timestamp and said each new day, resulting in the corresponding new, increased value for the index asymptotically increasing towards the starting value;
at the second timestamp, reducing a latest value of the index by the second dynamic claim metric; and
successively for each new day subsequent to the second timestamp, generating a different corresponding new, increased value for the index by dividing the third number of claims by a combination of the third number of days plus a second value that reflects a second number of days that have elapsed between the second timestamp and said each new day subsequent to the second timestamp, resulting in the different corresponding new, increased value for the index asymptotically increasing towards the starting value.
